(19) United States
(12) Patent Application Publication
(10) Pub. No.: US 2025/0263771 A1
(43) Pub. Date: Aug. 21, 2025

(54) SYSTEM AND METHODS TO MEASURE CELL VIABILITY IN HIGH THROUGHPUT VIA CONTINUOUS GEOMETRY

(71) Applicant: THE REGENTS OF THE UNIVERSITY OF COLORADO A BODY CORPORATE, Denver, CO (US)

(72) Inventors: Joel KRALJ, Louisville, CO (US); Christian Meyer, Longmont, CO (US)

(21) Appl. No.: 18/859,519

(22) PCT Filed: Apr. 25, 2023
(86) PCT No.: PCT/US2023/019799

§ 371 Date: Oct. 23, 2024

Related U.S. Application Data
(60) Provisional application No. 63/334,375, filed on Apr. 25, 2022.

(51) Int. Cl. C12Q 1/06; C12Q 1/24; G06V 10/26; G06V 20/69

(52) U.S. Cl. CPC C12Q 1/06; C12Q 1/24; G06V 10/26; G06V 20/693; G06V 20/695

(57) ABSTRACT

The present invention includes systems, methods, and compositions for a Geometric Viability Assay (GVA) adapted to measure individual colony-forming units (CPUs) from a diluted sample using one or more variable geometry vessels. The GVA system includes introducing a cell sample to a growth medium: utilizing an axially symmetric variable geometry vessel to incubate the cell sample in the growth medium: and utilizing an imager that is adapted to capture one or more images of the CPUs in the incubated variable geometry vessel.

### CROSS-REFERENCE TO RELATED APPLICATIONS

This International PCT application claims the benefit of and priority to U.S. Provisional Application No. 63/334,375 filed Apr. 25, 2022, the specification, claims and drawings of which are incorporated herein by reference in their entirety.

### STATEMENT OF GOVERNMENT INTEREST

This invention was made with government support under National Institutes of Health Grant No.'s 1DP2GM123458 and 5T32AG000279-14. The government retains certain rights in this invention.

### TECHNICAL FIELD

The present invention relates to high throughput systems for the objective, standardized determination of colony forming units using a cell detection system. Specifically, the invention is directed to a novel Geometric Viability Assay (GVA) adapted to measure individual colony-forming units from a microbial sample, and preferably a treated microbial sample, using one or more variable geometry vessels.

### BACKGROUND

The acceleration of antimicrobial resistance (A.MR) is precipitating a global health crisis. AMR was associated with an estimated 4.95 million deaths in 2019 and expected to result in over 10 million deaths by 2050. New techniques and innovations are urgently needed to expand the discovery pipeline for novel antibiotics and to identify antibiotic susceptibility more rapidly in clinical samples. Standard high throughput screens used in discovery pipelines use growth inhibition, commonly measured using optical absorbance, to quantify a compound's potential. But these assays are necessarily blind to slow-growing, drug tolerant persister cells, thought to be a major source of refractory clinical infections. Furthermore, growth inhibition cannot differentiate between bacteriostatic (i.e. stops growth) and bactericidal (i.e. induces cell death) compounds. Therefore, there is an urgent need for assays to identify compounds which are bactericidal, not merely bacteriostatic, in conditions which induce the physiology of persister bacteria (i.e. slow growing). This need is predicated on a rapid and scalable approach to measuring pathogen viability after drug treatment.

The gold standard in the field for measuring cell viability is the colony forming unit (CFU) assay. A core microbiology assay taught in undergraduate labs and used worldwide, the CFU assay uses a dilution series to quantify the number of cells capable of forming a colony. CFU assays have been used to quantify cell viability in systems including bacteria, fungi, hematopoietic progenitors, and cancer cells. The dynamic range of a CFU assay is routinely eight-orders of magnitude. This means in a population of one hundred million cells, a CFU assay can identify as few as one, or as many as one hundred million, resistant cells that are capable of surviving treatment and seeding recurrence. However, the CFU assay is time intensive and generates a significant amount of plastic waste. For the drop CFU assay, commonly 15 pipet tips per condition are required to run the 8-fold dilution and transfer to an agar pad. The time and cost has made it challenging to deploy the CFU assay in High-Throughput Screens (HTS). Prior approaches to address this fundamental problem either increased the speed by using robotics; decreased the amount of pipetting by using viability stains or droplet technologies; or used cell growth to estimate initial number of cells akin to qPCR. However, none of these approaches combines the simplicity of the CFU assay with its large dynamic range. As such, there exists a long-felt need for a simple, effective, and cost effective solutions to measure cell viability using a CFU assay. More specifically, a solution to the technical limitations outlined above would include an assay that: 1) can measure viability over several orders of magnitude; 2) is agnostic to cell growth rate; 3) requires one pipette tip per condition; and 4) is easy enough to scale to HTS without specialized equipment.

### SUMMARY OF THE INVENTION

In one aspect, the present invention describes an improved CFU assay, namely a Geometric Viability Assay (GVA) which leverages continuous geometry to run a dilution series with a high dynamic range in a single instance. As described herein, the systems, methods, and compositions of the GVA of the invention significantly simplifies and reduces the cost to measure cell viability over the traditional Colony Forming Unit (CFU) assay while maintaining comparable dynamic range and accuracy. GVA of the invention enables CFU assays at higher throughput and lower cost as compared to the traditional assay

In one preferred aspect, the GVA of the invention may include the generation of a sample of cells that are mixed with soft agarose and cast into one or more 3-D vessels having geometrically variable characteristics as described below. The probability of a colony forming at any position along the geometrically variable vessels is determined by the viable cell density and the vessel's three-dimensional shape. By calculating this probability, and by measuring the position of a subset of colonies in the vessel, the number of viable cells can be calculated with high precision. In this manner, the GVA of the invention simplifies and reduces the cost of measuring cell viability by a factor of fifteen-times (15×), can measure viability across more than 6 orders of magnitude, and enables more efficient drug discovery against clinically-relevant drug-resistant cells.

In another aspect, the inventive technology includes systems, methods, and apparatuses for a novel GVA as defined herein. In a preferred aspect, the system of the invention includes a growth medium for diluting a cell sample, and preferably a sample of prokaryotic or eukaryotic cells. The system of the invention may further include one or more variable geometry vessels, preferably being axially symmetric that are adapted to hold the diluted sample in the growth medium, and an imager adapted to capture one or more images of a subset of viable microbial CFUs present in the variable geometry vessel after an incubation period. In another preferred aspect, the cell sample can be treated, for example by an antibiotic or other experimental perturbation, prior to or within a variable geometry vessel of the invention.

In another aspect, the inventive technology includes systems, methods, and apparatus for screening novel compounds using the GVA of the invention. In this preferred aspect, a series of diluted samples of a target call, such as a prokaryotic or eukaryotic cells, can be treated with compounds from one or more drug or compound libraries and introduced into variable geometry vessels, preferably being axially symmetric that are adapted to hold the treated sample. An imager can be adapted to capture one or more images of a subset of viable CFUs present in the variable geometry vessel after an incubation period indicating the effect of the screened compound. In this manner, the GVA of the invention can quickly screen antibiotic sensitivity or resistance profiles from one or more target bacteria, as well as quickly determine minimum inhibitory concentrations (MIC) of the same.

In another aspect, the inventive technology includes systems, methods, and apparatuses for determining the microbial content, or load of a target object, such as a surface or biome using the GVA of the invention. In this preferred aspect, one or a series of samples are taken from a target object, such as the surface of a manufacturing facility, an environmental samples, a food or beverage product, or a biological sample from a subject, can be diluted and introduced into variable geometry vessels. An imager can be adapted to capture one or more images of a subset of viable CFUs present in the variable geometry vessel after an incubation period indicating the microbial load or biome characteristics present on the object. In one aspect, the GVA can be run prior to, and after a treatment, such as introduction of an antibiotic, or introduction of one or more bactericidal compounds, as well as cleaning or treating the target object, such as with traditional or anti-bacterial cleaners or UV light. In this manner, the GVA can be used to validate cleaning and sterilization protocols in commercial as well as therapeutic settings, such as hospitals and biological laboratories, as well as environmental locations and samples.

In another aspect, the inventive technology includes systems, methods, and apparatuses for diagnostic analysis of a biological or other sample using the GVA of the invention. In this preferred aspect, one or a series of biological samples subject or target object, can be diluted and introduced into variable geometry vessels, containing a diagnostic marker, such as a diagnostic agar or other compounds that can identify one or more pathologically relevant microbial species, such as human or animal pathogens. An imager can be adapted to capture one or more images of a subset of viable CFUs present in the variable geometry vessel after an incubation period indicating the presence of the pathogen, and further using the GVA to assist in the identification and diagnosis of the disease or condition, such as an infection with a clinically microbial or other organism.

In another aspect, the inventive technology includes systems, methods, and apparatuses for imaging a GVA of the invention. This system can include an imager configured to be positioned adjacent to a light source. In one preferred aspect, the imager comprises a smartphone, secured to a base and mechanically responsive to an adaptor. An axially symmetric variable geometry vessel can be used to incubate a cell sample in a growth medium and secured to a mount that can be coupled with the adaptor of the invention. In this configuration, the mount of the invention can be adjustable so as to position the vessel relative to the imager allowing it to capture one or more images of colony-forming units (CFUs) at the at the terminal portion of the incubated vessel. In alternative aspect, the adaptor can include a macro lens secured by a clip to facilitate the imaging of the vessel secured to the mount.

In another aspect, the inventive technology includes systems, methods, and apparatuses for imaging a GVA of the invention. This system can include an imager, such as a camera that can be adjustably positioned adjacent to a frame. In this embodiment, the frame of the invention can secure one or more axially symmetric variable geometry vessels adapted to incubate a cell sample in a growth medium. The frame and/or said imager of the invention are adjustable so as to position the vessel relative to the imager allowing it to capture one or more images of colony-forming units (CFUs) at the at the terminal portion of the incubated vessel.

In another aspect, the inventive technology includes systems, methods, and apparatuses for an imaging platform configured to capture one or more images of the vessel of the invention. In this embodiment, a processor can be responsive to an imager adapted to capture images of the images of colony-forming units (CFUs), preferably at the at the terminal portion of an axially symmetric variable geometry vessel of the invention. A computer executable program, responsive to the processor, can be adapted to identify one or more images of said CFUs at the terminal portion of said variable geometry vessel. In this aspect, the computer executable program can identify the boundaries of the tip of a variable geometry vessel, and further align the tip of the vessel. The computer executable program can further perform colony segmentation on the image, and from this colony segmentation further calculate the CFUs embedded in the growth media.

Additional aspects of the invention will be evident from the specification, figures, and claims provided herein.

### BRIEF DESCRIPTION OF DRAWINGS

FIG. 1A-I: The Geometric Viability Assay (GVA). a) The probability of a colony forming at a distance x from the tip of the cone is proportional to the infinitesimal volume dl (cyan circle) divided by the total volume I′ (purple cone). Analytically, this ratio is the Probability Density Function (PDF) as a function of x (see Supplemental Materials for derivation). b) The PDF for a cylinder (red), wedge (yellow), and cone (purple) as a function of the axial distance (x). c) Simulation of the colony distribution in a cone. d) Estimating the total CFUs/mL based on the position of colonies in the cone. (top) Shown are the distributions of colonies for 4 simulations spanning 20 to 10,000 CFUs/mL density. The volume of each cone is the same as in panel c. (bottom) GVA estimate of the CFUs/mL as a function of the included colonies and their x positions. e) The factor the GVA calculation differs from the correct value as a function of the number of colonies in Equation (1). Shaded error bars represent 1 standard deviation in 1000 simulations. Colors match simulations in panel d. f) Dilution series of E. coli embedded in 150 μL 0.5% LB-agarose in p200 pipette tips. Red circles correspond to colonies counted using a custom semi-automated segmentation software. g) E. coli CFUs/mL calculated using GVA for a 4× dilution series. Points are the mean of 4 replicates. Mean calculated after taking the log. Red line is the linear regression fit to dilution series. A slope of 1 on a log-log plot is expected if the GVA estimate scales linearly with dilution. h) The drop CFU and GVA estimates are significantly correlated over 6 orders of magnitude. i) GVA performed on gram-positive, gram-negative, and eukaryotic cells (see FIG. 10a for quantification)

FIG. 2A-D: GVA dynamic range, but not accuracy, depends on the optical configuration. a) Picture of assembled pipette tip holder on an iphone 12 with a Xenvo macro lens. The pipette images are taken in front of a white backdrop (paper) with ambient illumination. b) Example images of the same 2 pipette tips using the Canon EOS with 100 mm f2.8 macro lens (left) or the iPhone 12 with Xenvo macro lens (right). The GVA calculated CFUs/mL are reported below. Selected colonies for GVA calculation are circled. c) Dynamic range of the iPhone GVA. E.

coli were diluted 4X and embedded in pipette tips. After incubation, the same tips were imaged with the iPhone camera with macro lens (green) and the mirrorless camera (purple). Points are the mean of 4 replicates calculated after taking the log. Green and purple lines are the linear regression fit to the dilution series. d) Pearson correlation between iPhone GVA and professional camera for all pipettes where colonies could be counted using both. Correlation coefficient calculated in log-space.

FIG. 3A-J: GVA reduces the time and materials of viability measurements by over 10-fold. a) (left) Schematic of a drop CFU assay and required materials for 96 samples assuming tips are changed for each dilution step. (middle) A Spiral Plater spreads a sample in an Archimedes spiral on a solid media plate. The spiral results in decreasing sample volume as a function of radial distance with a reported 3-log dynamic range. One petri dish is required per sample. (right) GVA uses a single pipette tip to run a 6-order dilution series. b-d) Time comparisons for different techniques. b) Time required to prepare solid growth media. The preparation time for the Spiral Plater and drop CFU includes: 1) autoclaving the agar; 2) cooling post autoclave; 3) plate pouring; and 4) an plate cooling. GVA melts agarose in a microwave which is subsequently equilibrated in a warm bath for 1 hour prior to starting. c) Sample plating from a 96-well plate. Time for the Spiral Plater assay sample plating based on industry-reported value. Drop CFU was timed by an expert user using a 12-channel pipette and changing tips at every dilution and plating step. d) Time required for quantification of 96 samples. Spiral Plater time is based on industry-reported value using an automated colony counter. GVA time includes imaging (7 min for Canon with motorized stage and 30 min for iPhone), image preprocessing and tip segmentation (5 min), and semi-automated colony counting (10 min) for 96 pipette tips. The drop CFU colonies were counted and recorded manually. e) Number and cost of pipette tips as a function of sample count for the three different techniques. See Supplemental Table 1 for cost estimates. f) Amount of agar required as a function of sample count. 25mL of 1.5% agar per 15 cm petri dish was assumed for the drop CFU and Spiral Plater assays. 200 μL of 0.5% agarose per tip was assumed for the GVA. g) Number of 96-well and petri dishes per condition. h) Estimated total cost in consumables per 96 samples of the three methods. GVA cost is $0.17/sample. i) Instrument costs. Based on quotes for a Spiral Plater (SP) and automated imaging system from 3 manufacturers. GVA instrument cost included the Canon camera and 100 mm f/2.8 macro lens. j) The difference in instrumentation cost for the Canon and iPhone optical configurations.

FIG. 4A-H: GVA has a low noise profile and is robust to missing colonies or tip position errors. a,b) Coefficient of Variation (COV) between 4 technical replicates for different number of CFU concentrations for GVA using the Canon or iPhone optical configuration (a) and drop CFU (b). c,d) The factor the GVA calculation differs from the correct value as a function of the number of missed colonies (c) or error in tip position (d) in simulated results (see Methods).

Shaded error bar is the standard deviation in 1000 simulations. e,f) Same error calculations for experimental data. Error bars represent the standard deviation between all the pipette tips (#) included in each bin. g,h) Correlation between the GVA and the drop CFU assay as a function of counting and position errors.

FIG. 5A-H: GVA screening of the Enzo library identifies DPI as active against stationary phase E. coli. a) Dose-response of 3 antibiotics for stationary and exponential (ex) cultures after 24 hours of treatment. Each point is the mean of duplicate measurements. CFUs/mL were normalized to an untreated control. b) Viability over time for stationary and exponential cells at one concentration of antibiotic. c) Drug classes of the Enzo Bioactive Screening Library. The size of donut wedge is proportional to drug class representation. Targets of each class and relative representation depicted on the outer ring. d) Absolute viability of stationary (green) and exponentially growing (purple) cells after 24 hours of treatment with Enzo library. Each condition was run in duplicate and the mean taken in log-space. e) Scatter plot of stationary phase versus exponential phase from the screen. The standard deviation of DMSO controls are depicted with a red cross. Selected hits are annotated. f,g,h) Mitomycin C (DNA crosslinker), phentolamine (α-adrenergic antagonist), and DPI (NADPH oxidase inhibitor) dose responses in stationary and exponential cultures.

FIG. 6A-I: DPI generates ROS, activates the SOS response, and antagonizes ciprofloxacin. a) Median, single-cell CellROX signal as a function of time for DPI (blue), ciprofloxacin (orange), and an untreated control (yellow). b) Efficacy of DPI in aerobic and anaerobic conditions. See FIG. 18b,c for ciprofloxacin and gentamicin. c) Images of live E. coli cells stained with the CellROX dye for three DPI concentrations 4 hours after adding DPI. Brightness and contrast is the same for all images. See Supplemental Movie 2. d) Measurement of polB and rrnB promoter activity normalized to t=0. e) DPI dose response for E. coli knockout mutants treated during stationary (top panels) or exponential growth (bottom panels). The dose response for the wild-type (WT) cells is depicted in green or purple, respectively. Shaded errorbars equal to the standard deviation in logspace between 3 replicates. See FIG. 20 for other mutants. f) GVA checkerboard assay for DPI combined with ciprofloxacin at 24 hours. Each square in the heatmap was the mean of duplicate conditions. Colorbar correspond to the log10 (CFUs/mL) for each dose combination. Left panel shows the dose response for DPI plus 1 μg/mL ciprofloxacin (cyan). See FIG. 21 for full time series. g) GVA checkerboard assay for DPI combined with gentamicin at 24 hours. h) Growth inhibition checkerboard for DPI and ciprofloxacin. Optical density was measured for each condition over 8 hours and the integrated area under the growth curve (AUGC) is depicted (colorbar). i) Dose response curves for temporally staggered combinations. All treatments lasted for 24 hours total. Pretreated conditions were treated for 2 hours with a single drug followed by 22 hours with both drugs.

FIG. 7A-F: Derivation of a cone's PDF. a) The volume of the infinitesimal dV divided by the total volume V corresponds to the probability of finding a colony as a function of x. The radius of the infinitesimal (r′(x)) is a function of the radius of the cone's base (r) divided by the height of the cone (h) times x according to trigonometry. b) The PDF of the cone as a function of x. Overhead projection of cone is depicted above. c) The cumulative density function (CDF) as a function of x. d) The PDF is the same for axially symmetric cones such as a square (red) and triangle (turquoise) pyramids. e) Two equivalent ways of calculating the number of CFUs in the wedge using either the CDF (left) or PDF (right). N (x) is the number of colonies counted. f) Percentage of simulations with the GVA calculated CFUs/mL within a factor of 2 of the correct value as a function of the number of colonies used for the GVA calculation. 1000 simulations were used to calculate percentage. See FIG. 1c for simulation parameters.

FIG. 8A-C: Optical configuration. a) Schematic of optical configuration for imaging the pipette tips containing agarose. A mirrorless camera with a macro lens is positioned above the tips at the focal plane. The addition of a z-positioner stage helps in fine tuning the focus. Pipettes are illuminated transversely using an LED light box with a diffuser. This box is mounted on a stepper motor to allow for imaging 12 pipettes at a time. The stepper motor and the camera are simultaneously controlled via Lab View software. b) Picture of optical configuration. A cyan light was used to maximize the contrast of the TTC counterstain. A styrofoam box functions as a reflective light box and a sheet of paper as a diffuser. The GVA samples are positioned using a 12 channel pipetter and imaged using a Canon EOS RP camera with a f/2.8 100 mm macro lens. c) Pixel resolution for this configuration is 6.7 microns.

FIG. 9: Example drop CFU plate. a) Each condition (columns) is diluted with a 10-fold serial dilution (rows) and 3 μL are spotted on a 1.5% LB agar pad poured into an empty tip box. Colonies are counted for the dilution row where individual colonies are discrete. These counts are used to calculate the CFUs/mL (bottom).

FIG. 10A-D: GVA calculations for different species. a) For the six species tested with GVA, the estimated number of CFUs/mL for different dilution series. b) Plates streaked with pipette tip after GVA embedding before or after bleach wash. No change in CFUs/mL were observed after bleach wash. c) Example GVA pipette tip for an E. coli biofilm. See Methods for culture and dissociation protocol. d) Biofilm growth over time. Error bars correspond to standard deviation between ≥5 biological replicates.

FIG. 11A-C: Biome sampling using GVA. a) Twenty-four positions (red dots) on a volunteer were swabbed vigorously for 15 seconds before being placed in 1 mL of LB medium and vortexed for 10 seconds. 50 μL of the sample was then mixed with 150 μL of 0.66% melted LB agar to a final concentration of 0.5% agar and allowed to gel in the tips. With this protocol, the lower limit of detection was 20 CFUs/mL (dotted line). The sample replicates were incubated at 30° C. or 25° C. for 48 hours before imaging. b) Example pipette tips for different sample regions reveals diverse colony structure and concentration for different biome locations. All samples were stained with TTC. c) Samples from higher thermal regions (ear, armpit) grew at 30° C. but did not grow at 25° C. indicating the temperature selectivity of different species grown in the pipette tip.

FIG. 12A-J: Chip version of GVA uses the square pyramid geometry. a,b) 3D printed molds for creating square pyramid for 12 (a) and 48 (b) conditions. c) Picture of a 9× dilution series of E. coli cultures on the GVA chip. d) GVA calculated CFUs/mL using for a dilution series. Each dot is the mean of 4 replicates. c) The noise, measured using the coefficient of variation (COV) for the chip GVA. f) Matched drop CFU quantification to conditions in (d). g) Corresponding noise analysis for drop CFU. h) Correlation between chip GVA and drop CFU over 5 orders of magnitude. i,j) Chip GVA for gram-positive (i) and eukaryotic (j) cells.

FIG. 13: Smartphone (iPhone) pipette tip holder. a) The 3D printed parts for stereotypically positioning a pipette tip in front of an iPhone rear camera with a Xenvo macro lens (15× magnification without the widefield lens). The blue face plate slides onto the Xenvo macro lens which is clipped to the iPhone. The green bar is attached with a screw to the side channel on the blue plate. This allows for adjusting the height by sliding the green bar in the channel. The purple extension bar slides into the green channel to adjust the imaging depth. The smartphone is held upright with a stand (yellow). Pieces printed with standard FDM printing with PLA.

FIG. 14A-C: Sensitivity analysis of GVA calculations to error in missing colonies and location of the tip. a) Heatmap of the error as a function of both tip position and missing colony errors. b) Same analysis as in panel a, but with experimental data. CFUs/mL binned between 1e3 and 1e5 (top row), 1e5 and 1e7 (middle row), and 1e7 to 1e9 (bottom row). The number of pipette tips included in each bin is annotated by the count. c) Heatmap of the Pearson correlation between the drop CFU and GVA for both tip position and missing colony errors.

FIG. 15A-B: Cell counts over time in stationary versus exponential cultures. a) Number of CFUs/mL in stationary (a) versus exponential culture (b). To generate exponential culture, stationary phase cells were diluted 1:1000 in fresh LB media and place in the shaking incubator (180 RPM) at 37° C. for 2 hours prior to beginning the experiment.

FIG. 16A-C: Enzo screen controls. a) Library diversity of ICCB Enzo Known Bioactive library compared to the Maybridge HitFinder library. Tanimoto similarity between all molecules based on SMILES was calculated using the RDKit package in python. From this distance matrix, the tSNE embedding was initialized with PCA and computed with a perplexity of 50. b) Distribution of CFUs/mL for conditions on the edge of the plate versus in the center wells for both stationary and exponential cultures. Statistical test used a Mann-Whitney U test for nonparametric distributions (p-val 0.05). c) Distribution of CFUs/mL for different drug classes identified in the Enzo Library (See FIG. 5c). No class differences were found when using ANOVA (p-val 0.001, p-val corrected for multiple hypothesis testing). No differences from control were found using the Pairwise Tukey Test (p-val 0.01, Pairwise Tukey Test)

FIG. 17: Non-validated hits from the ICCB Enzo bioactive screen. E-4031 (a) and phenamil (b) dose-response curves against stationary or exponentially (ex) growing cultures.

FIG. 18A-D: a) Duration of ROS reduction and onset of the secondary ROS spike is DPI-concentration dependent. Depicted is the median single-cell CellROX signal as a function of time for different concentrations of DPI. b,c) Dose response curve for ciprofloxacin (b) and gentamicin (c) against stationary phase cells in aerobic or anaerobic conditions. Treatment was for 24 hours. d) Efficacy of DPI as a function of increasing concentrations of the ROS-scavenger, ascorbic acid (AA).

FIG. 19: Strip charts of lexA-repressed genes (rows) using the PEC library. GFP fluorescence (top panels of each row) is proportional to each gene's promoter activity. The bottom panel of each row depicts bright field image. Columns correspond to different timepoints post treatment.

FIG. 20: Sensitivity of gene mutants to DPI in exponential and stationary phase. Wild type reference depicted in solid line for each mutant. Error bars are the standard deviation in log space between three biological replicates. Mutants were selected from the Keio collection. Kanamycin (25 μg/mL) was included in the all Keio culture conditions both in the overnight culture and during treatment with DPI to maintain gene knockout.

FIG. 21: GVA temporal checkerboard of DPI crossed with either ciprofloxacin (left panels) or gentamicin (right panels) against E. coli. Treatment time increases down the rows. Each square in the heatmap was the mean of duplicate conditions. Colorbar correspond to the measured log10 (CFUs/mL) for each combination. Left panel shows line trace (cyan) for the DPI dose response at 1 μg/mL ciprofloxacin or 10 μg/mL gentamicin.

FIG. 22: GVA temporal checkerboard of DPI crossed with either ciprofloxacin (left panels) or gentamicin (right panels) against S. typhimurium.

FIG. 23: shows a schematic of the procedure for a standardized method for a traditional CFU assay using a drop plate method. Scaling to 96 measurements, and assuming a standard 12 channel pipet, this traditional CFU requires 1440 pipet tips, approximately ˜50 min to set up and compete, and at least 8 agar plates.

FIG. 24: shows a schematic of the GVA of the invention, with optionally multi-vessel plate. Scaling to 96 measurements. Scaling to 96 measurements, and assuming a standard 12 channel pipette, GVA of the invention requires 96 pipet tips, approximately ˜6 min, to set up and compete, in a preferred embodiment 1 to two 2 reusable chips,

FIG. 25: shows a schematic of an alternative embodiment GVA of the invention, wherein the variable geometry vessel includes a pipette tip. Scaling to 96 measurements. Scaling to 96 measurements, and assuming a standard 12 channel pipet, GVA of the invention requires 96 pipet tips, approximately ˜6 min.

FIGS. 26A-B: Shows the quantified data from the example shown in FIG. 10. (a) estimated CFUs as a function of dilution. (b) 3× replicate data showing mean CFUs.

FIGS. 27A-B: Screen of compounds that affect viability in exponential and stationary phase using data from a GVA assay in 96 well format drug testing. The antibiotic potential of a drug library (80 compounds) was tested against exponential and stationary phase bacteria in duplicate. (A) Viability reduction of compounds in exponential (top) and stationary (bottom) phase cells. Each x is the mean of 2 biological replicates. (B) Scatter plot of the viability screen comparing exponential and stationary phase activity. Each blue dot represents the mean of 2 replicates of a compound treatment. Red dots represent DMSO controls. The dashed lines represent 3 times the standard deviation of the negative controls. Screen was performed with E. coli with both stationary phase and exponential phase bacterial populations. Both phases were screened in duplicate. 80 compounds and 16 controls were screened. Cells in stationary and exponential phase were treated with the compound for 4 hours, followed by casting into pipet tips using the GVA protocol. Two compounds, mitomycin C and diphenyeneiodium both showed bactericidal activity.

FIG. 28A-B: Shows the utility of the GVA assay for calculating minimum inhibitory concentration (MIC). (A) Images of GVA measured cells at a range of kanamycin concentrations within the agarose matrix. 1×10¹⁰ cells/mL were embedded in each sample along with the specified amount of kanamycin. The calculated CFUs from the GVA assay are shown as well. (B) Plot of biological triplicate samples with kanamycin treatment. The dashed line shows the detection limit of the assay for kanamycin.

FIG. 29: GVA can accurately estimate CFU in a rapid timeframe. The images show the same pipet tip imaged after 4, 6, 8 and 24 hours incubation at 37 C. The colonies are clearly visible after 6 hours, and counting at 8 hours showed up to 7 orders of dynamic range with high accuracy (bottom heat map). For use in rapid antibiotic testing, these measurements can be acquired within 8 hours without a necessary pre-incubation step.

FIG. 30A-B: (A) Estimating the total CFUs/mL based on the position of colonies in the cone. (top) Shown are the distributions of colonies for 4 simulations spanning 20 to 10,000 CFUs/mL density. The volume of each cone is the same as in panel c. (bottom) GVA estimate of the CFUs/mL as a function of the included colonies and their x positions. (B) The factor the GVA calculation differs from the correct value as a function of the number of colonies in equation (1). Shaded errorbars represent 1 standard deviation in 1000 simulations. Colors match simulations in panel A.

FIG. 31A-B: (A) GVA works with Gram-positive (top), Gram-negative (middle) and eukaryotic (bottom) cells. (B) A serial dilution of S. cerevisiae (baker's yeast) grown in YEPD to determine the dynamic range with eukaryotes. GVA provides accurate estimate of yeast viability up to almost 7 orders of magnitude.

FIG. 32A-B: (A) An image of the active region of the software based colony counting software. The tip is marked with a red vertical line, and individual identified colonies are marked with red circles. The current CFU count is shown on the top. (B) The setup of the software algorithm to define the experimental parameters (left) as well as the location of the images (middle). The user can also fine tune the colony segmentation algorithm (right) or accept the default parameters.

FIG. 33: Flow chart of the GVA assay. The physical (hardware) based measurements involve positioning the sample in front of a measurement device, followed by images of the tips. Within the software, a user identifies individual pipet tips, aligns them to an orthogonal plane for easy distance calculation, selects a subset of colonies within the tip, and then uses the algorithm to estimate the number of CFUs in the entire tip.

FIG. 34A-C: One instantiation of using the GVA assay with a paper based readout (no imaging system required). (A) The calculation for estimating CFUs and a ruler assuming a 36 mm tip with a agarose volume of 150 μL The image shows a serial dilution of bacteria overlaid on the paper based ruler. The CFU estimate is based on the location of the 10^{th} colony counted by the user. (B) Comparison between CFU measurements of GCA with a high resolution Canon camera and macro lens (purple) compared to the paper method (green) using a basic magnifying glass. The accuracy is identical with the paper, but the maximum number of resolvable colonies is reduced. A comparison between paper and camera systems (bottom) shows a very high correlation over the dynamic range of the paper based measurement.

FIG. 35: Minimum inhibitory concentration (MIC) measurements are independent of the starting concentration of bacteria. Each box represents GVA measurements with increasing amounts of antibiotic (x-axis). The number of viable cells in each concentration is plotted on the y-axis. The antibiotic is labeled on the top. Within each box, each color represents the initial cell population, from 1000 to 1,000,000 cells per milliliter. The MIC is identical for each drug regardless of the starting concentration of cells.

FIG. 36: GVA calculates the minimum inhibitory concentration for diverse bacterial species. Each box represents the number of CFUs as a function of increasing antibiotic concentration indicated on the x-axis. The antibiotic identity is printed on the top of each box. Each color represents a different bacterial species. The different MICs indicate that each species has a unique antibiotic susceptibility spectrum that is easily revealed by GVA.

FIG. 37: GVA can rapidly measure minimum inhibitory concentration for diverse bacteria. Each box represents the number of CFUs as a function of increasing antibiotic concentration indicated on the x-axis. The antibiotic identity is printed on the top of each box. Each color represents a different bacterial species. These measurements were taken after 12 hour incubation with consistent measurements after 24 and 48 hours.

FIG. 38A-B: GVA imaging works in blood-agar, a common medium to grow pathogenic strains. (A) Image of E. coli embedded in blood agar at the manufacturers recommended concentrations and grown overnight at 37 C. (B) Quantification of a serial dilution of CFUs. Over 5 orders of magnitude are resolvable in this medium, similar to results with LB or minimal medium.

FIG. 39A-B: Example use cases enabled by GVA. (1) High-throughput viability screens. The Prestwick library of compounds (1440) was run in duplicate (2880 total CFU measurements) in ˜2 weeks. This screen can help identify new antibiotic compounds or combinations. (2) Pharamcokinetic and pharmacodynamic characterization of antibiotics. Antibiotic effects were measured as a function of time, concentration, and bacterial species. (3) Drug combination matrices. Checkerborards comparing a range of compound 1 and compound 2, as a function of time. Combinations were measured for multiple compounds and times. (4) Pharmacogenomic characterization of antibiotic efficacy. Measuring viability across multiple concentrations of a compound against multiple genomic bases. The effects were measured across multiple genomic modifications and antibiotic concentrations.

FIG. 40: Effects of potential errors on counting accuracy. (Left) Change in the Pearson r coefficient as colonies are not included in the count. The total count was 30 colonies. The correlation changes less than 1%. (Right) Change in GVA accuracy if the tip position is improperly assigned in the software. The PCC changes by less than 1% when missing the tip position by up to 4 mm on a 36 mm pipet tip.

FIG. 41: shows a front perspective view of a GVA assay imaging system in one embodiment thereof.

FIG. 42: shows a front perspective view of a frame for a GVA assay imaging system in one embodiment thereof.

FIG. 43: shows a front perspective view of a imager bracket for a GVA assay imaging system in one embodiment thereof.

FIG. 44: shows a front perspective view of a mounting block for a GVA assay imaging system in one embodiment thereof.

FIG. 45: shows a front perspective view of a plate for a GVA assay imaging system in one embodiment thereof.

FIG. 46: shows a top perspective view of a GVA assay imaging system utilizing a smart phone mechanically responsive to an adaptor in one embodiment thereof.

FIG. 47: shows a front perspective view of a GVA assay imaging system utilizing a smart phone mechanically responsive to an adaptor in one embodiment thereof.

FIG. 48: shows an adaptor for a GVA assay imaging system having a vertical adjustor in one embodiment thereof.

FIG. 49: shows a depth adjustor for a GVA assay imaging system in one embodiment thereof.

FIG. 50: shows a mount having a vessel holder for a GVA assay imaging system in one embodiment thereof.

FIG. 51: shows a base for a GVA assay imaging system in one embodiment thereof.

### DETAILED DESCRIPTION OF INVENTION

The present invention includes a novel viability assay, generally referred to herein as the Geometric Viability Assay (GVA). In a preferred embodiment, the GVA of the invention calculates the CFUs in a sample, such as a biological, environmental or commercial sample, based on the axial position of embedded colonies that form in an axially symmetric variable geometry vessel, which in a preferred embodiment includes cone-shaped vessel as described herein. Based on the physical characteristics of the axially symmetric variable geometry vessel the probability of a colony forming at the tip of the vessel is less than near the base. Analytically, this probability is proportional to the squared perpendicular distance of the colony to the vessel tip. By measuring the position of a limited number of colonies in the vessel and utilizing the derived probability density function, the total number of colonies in the entire vessel can be computed with high precision.

By leveraging the latent information encoded in the colony distribution, the GVA of the invention can accurately quantify the number of viable cells in a sample ranging from 1 cell to 10,000,000. This dynamic range can be accomplished using a cone-shaped axially symmetric variable geometry vessel universal in microbiology-the pipette tip. As further described below, the GVA of the invention: 1) measures viability over 6 orders of magnitude; 2) does not depend on the cell's growth or lag phase; 3) minimizes consumables; and, 4) reduces operator time by over 30-fold compared to a traditional drop CFU assay. Combined the GVA of the invention enables throughputs of up to 2000 viability measurements per researcher per day. The inventive technology includes a novel system for a colony-forming CFU assay. In a preferred aspect, the device of the invention includes a variable geometry vessel adapted to hold a diluted sample in a growth medium. As used herein, a “sample” or “cell sample” of the invention can include a sample containing one or more cells to be cultured tested. In a preferred embodiment, the a “cell” or “cell to be cultured or detected” can include both prokaryotic and eukaryotic cells that can be culture and be identified as a CFU, and can preferably include gram positive and Gram negative bacteria, as well as fungal, yeast, or even algal cells. In another preferred embodiment, a “cell” can include a pathogenic bacterium, or a complex cellular sample, such as a biome sample, or other sample from a surface or object, such as a food or beverage containing a plurality of different cells.

In another preferred embodiment, the a “sample” or “cell sample” of the invention can include a biological sample. As used herein, the term “biological sample” includes a sample from any bodily fluid or tissue. In certain embodiments, a biological sample can include samples that are typically subject to clinical or diagnostic testing. Biological samples or samples appropriate for use according to the methods provided herein include, without limitation, blood, serum, urine, saliva, tissues, cells, and organs, or portions thereof. A “subject” is any organism of interest, generally a mammalian subject, and preferably a human subject.

In another preferred embodiment, the “sample” of the invention can include an “environmental sample.” As used herein, an “environmental sample” means a sample taken or acquired from any part of an internal or external environment. In a preferred embodiment, an “environmental sample” can include a sample from a water, soil, municipal waste, hazardous waste, potential pollutants and others. In another preferred embodiment, an “environmental sample” can include a “commercial sample” which can include a fomite, such as a surface of a commercial manufacturing facility, or an object, such as a food and/or beverage and the like.

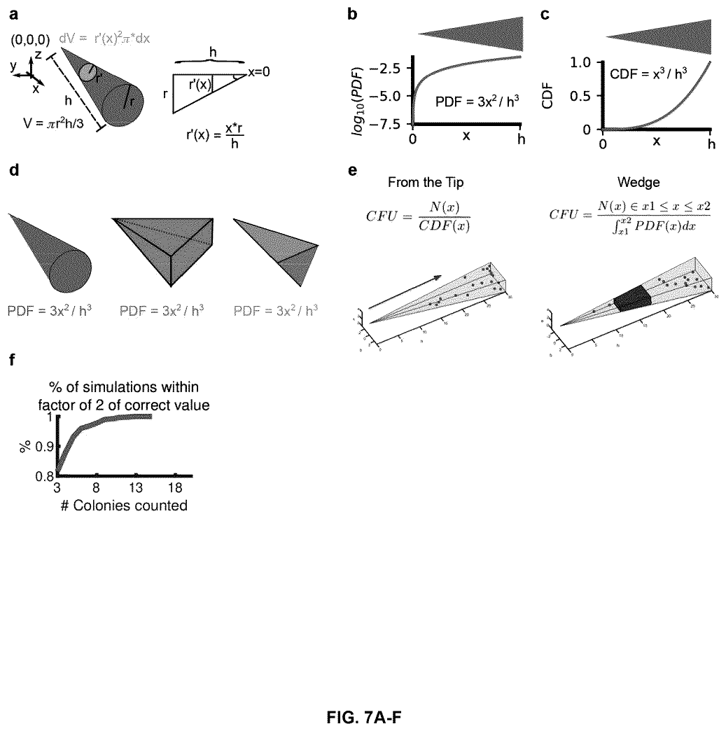

Referring to FIG. 7, a variable geometry vessel of the invention includes an opening to permit disposition of the growth medium, and an intermediate portion adapted to have a geometric configuration that changes size across all three-dimensions, and a terminal portion being narrower than said opening. The device of the invention may include a plurality of variable geometry vessels, for example produced in a mold having one or more variable geometry vessels, or a plurality of vessels positioned adjacent to one another to allow rapid comparison and analysis. Specifically, as used herein, a “variable geometry vessel,” means an axially symmetric vessel or container adapted to hold a quantity of growth medium wherein the dimension of the vessel changes size across all three-dimensions. Notably, the vessel changing size across all three-dimensions can be continuous, or non-continuous in certain embodiments. Examples of axially symmetric variable geometry vessel configurations that change size across all three-dimensions, include but are not limited to the following shapes shown in FIG. 7D:

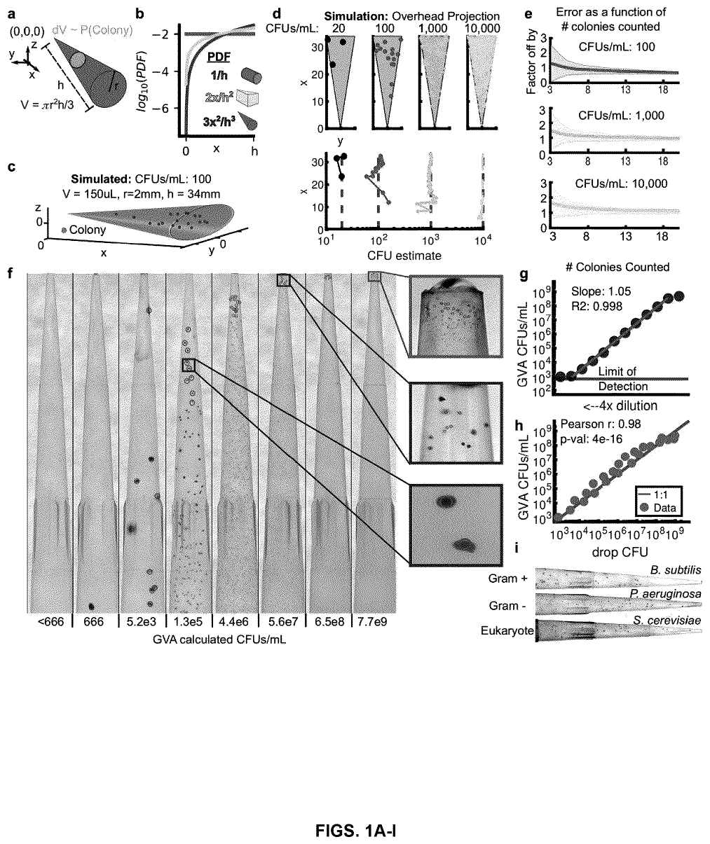

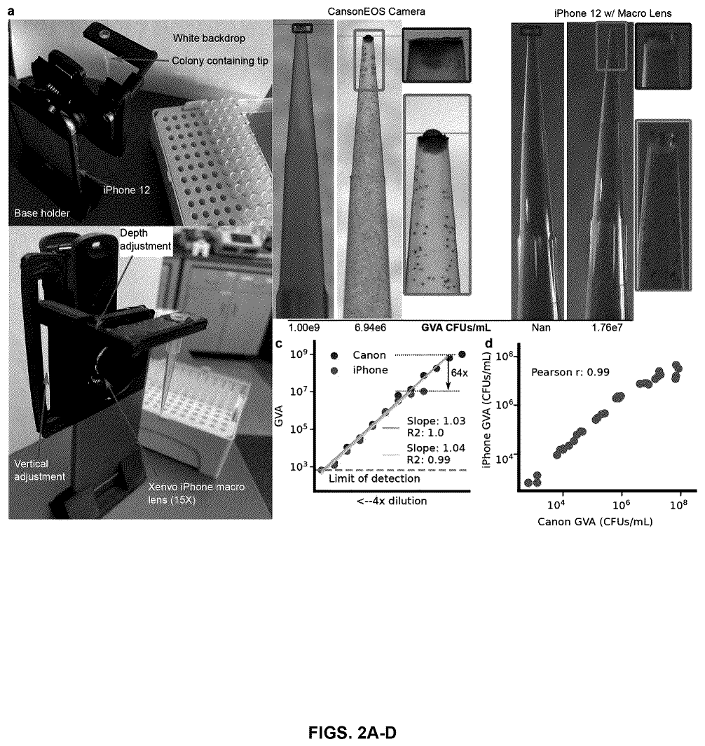

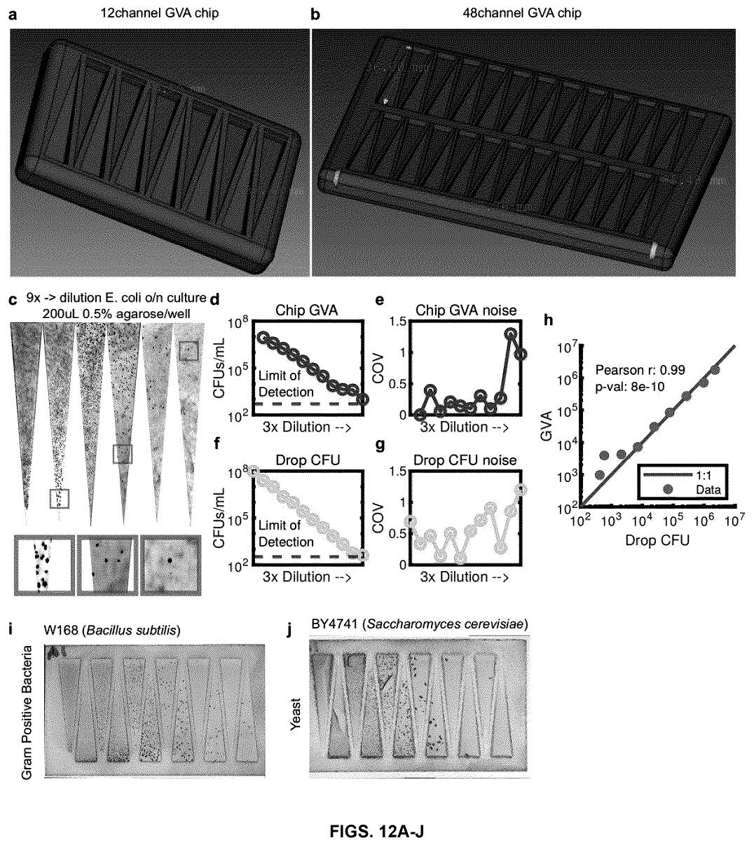

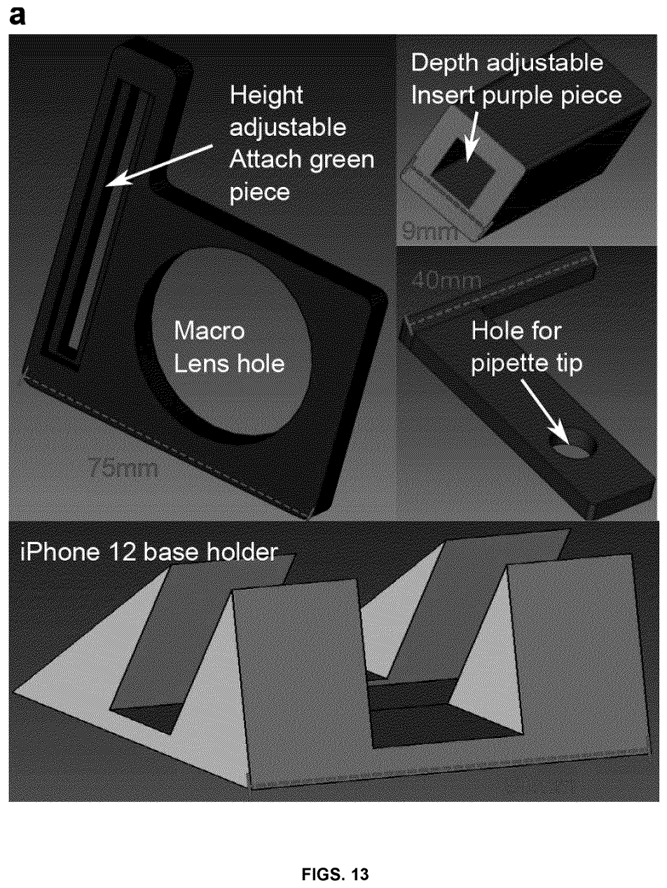

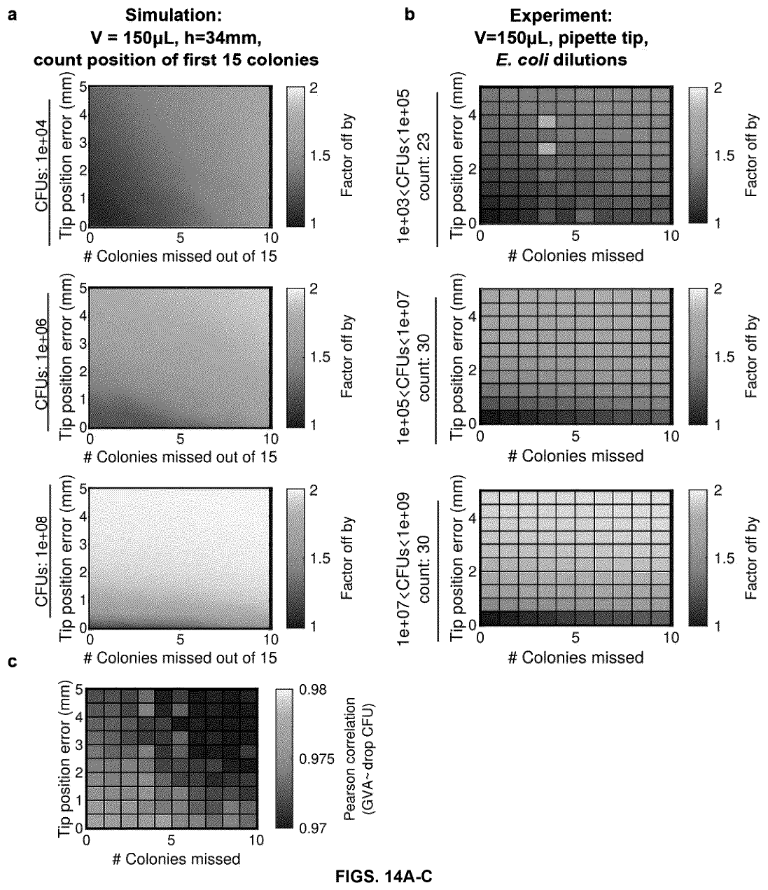

As shown in FIG. 1f, and 2b in one embodiment an variable geometry vessel of the invention can include a standard translucent pipette tip or cone that would be recognized by one of ordinary skill in the art. Again, as shown in FIG. 2, one or more pipette tips that can be coupled singly, or in a plurality with a standard multi-use laboratory pipette, or pipette holder as discussed below. As further shown in FIG. 12a-b, in one embodiment, a variable geometry vessel of the invention can include a mold having one or more variable geometry vessels. The mold may include a three-dimensionally-printed chip having a plurality of variable geometry vessels positioned adjacent to one another. As noted above, the variable geometry vessels of the invention can preferably be translucent to allow direct image capturing of all or a portion of the colonies formed within the growth medium.

The inventive technology includes novel systems and methods of establishing a geometric viability assay (GVA). As used herein, “GVA,” describes a CFU assay performed using a variable geometry vessel as described herein. In a preferred aspect, the GVA of the invention may include cell sample, preferably a biological, environmental or commercial sample containing one or plurality of cells that can be cultured. In a preferred embodiment, the cell can include a bacterial, yeast or fungal cell that can be cultured in a liquid culture media to a specific density. In a preferred embodiment, this sample may further be treated. As described herein, the term “treated” includes the step of subjecting the cell to an experimental perturbation (e.g. drug treatment, culture condition, genetic modification and the like) to be measured. In a preferred embodiment, the sample is treated with one or more compounds that are adapted to kill and/or inhibit the growth of the sample and/or effect one or more phenotypic or genotypic changes in said sample. In a preferred embodiment, such treatment may include one or more compounds selected from the group consisting of: a therapeutic compound, an antibiotic, a bactericidal compounds, a bacteriostatic compounds, an anti-cancer-compound, an anti-fungal agent.

In one embodiment, a sample can be diluted in a growth medium and positioned within a variable geometry vessel and incubated. For example, in one preferred embodiment, a sample may be diluted to a desired concentration of CFUs, directly in a growth medium, such as a quantity of melted liquid agarose, and preferably 0.5% liquid agarose which can further be cooled to ˜37 C. to allow the material to be cast into a variable geometry vessel. Naturally, various liquid and solid growth media for prokaryotic and eukaryotic cells would be known in the art and adaptable to the GVA of the invention.

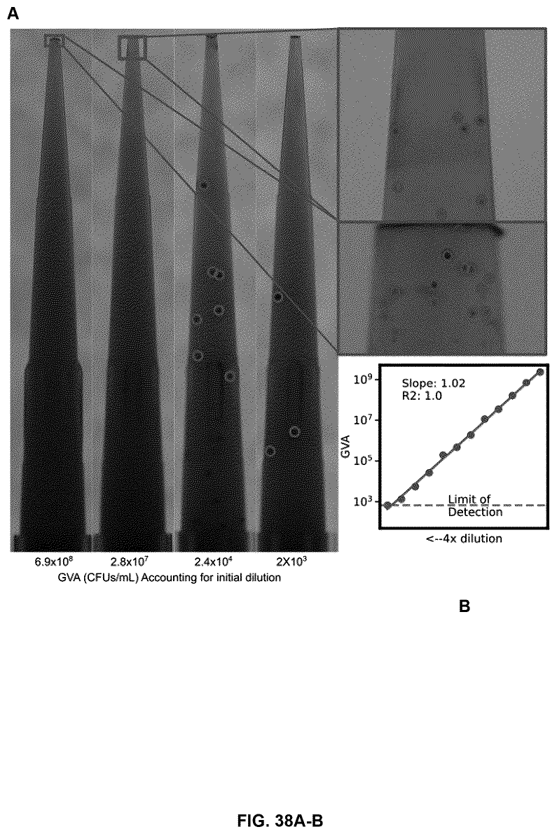

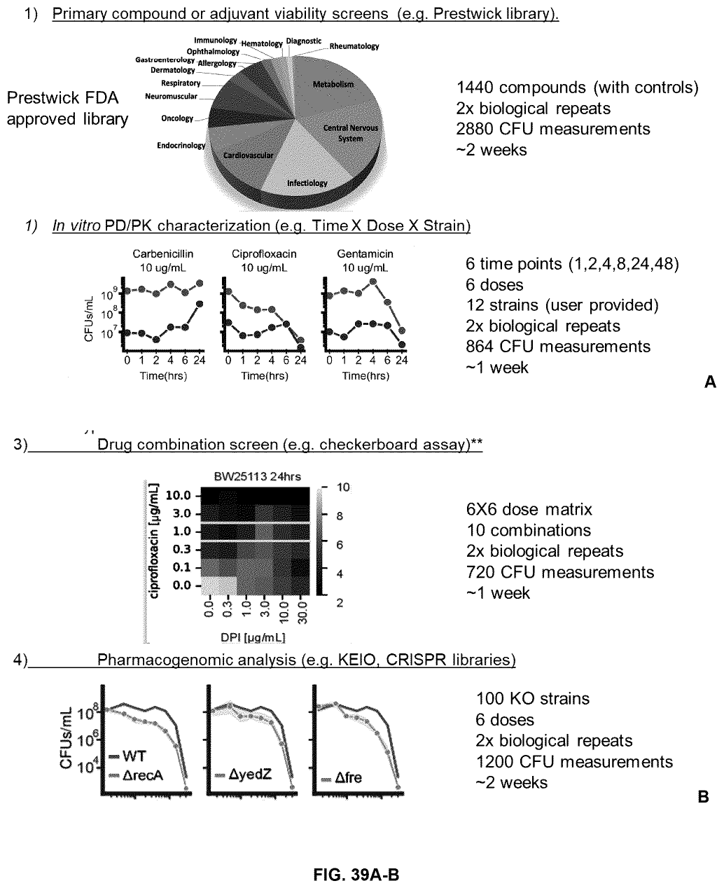

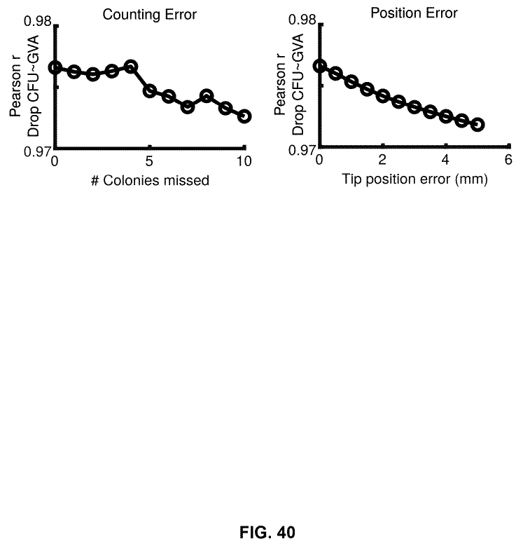

In one preferred embodiment, a sample containing one or a plurality of different microorganisms, can be taken from a sample, or isolated and grown from a sample, and further diluted in a growth medium, such as nutrient agar, and the like, which is capable of allowing the formation of a colony from a microorganism. In this embodiment, one or more contrasting agents, such as the microbial stain 2,3,5-Triphenyltetrazolium chloride (TTC) or a cell permeable fluorescent dye can be added to the growth medium to help with later CFU visualization as describe below. In alternative embodiments, the growth medium can include a clinical or diagnostic indicator. For example, as shown in FIG. 38, a sample containing one or a plurality of different microorganisms, can be taken from a sample, or isolated and grown from a sample, and further diluted in a diagnostic growth medium, such as blood agar, which is capable of allowing the formation of a colony from a microorganism. As shown, blood agar is an enriched medium used in clinical and diagnostic setting to grow fastidious organisms and to differentiate various bacteria based on their hemolytic properties.

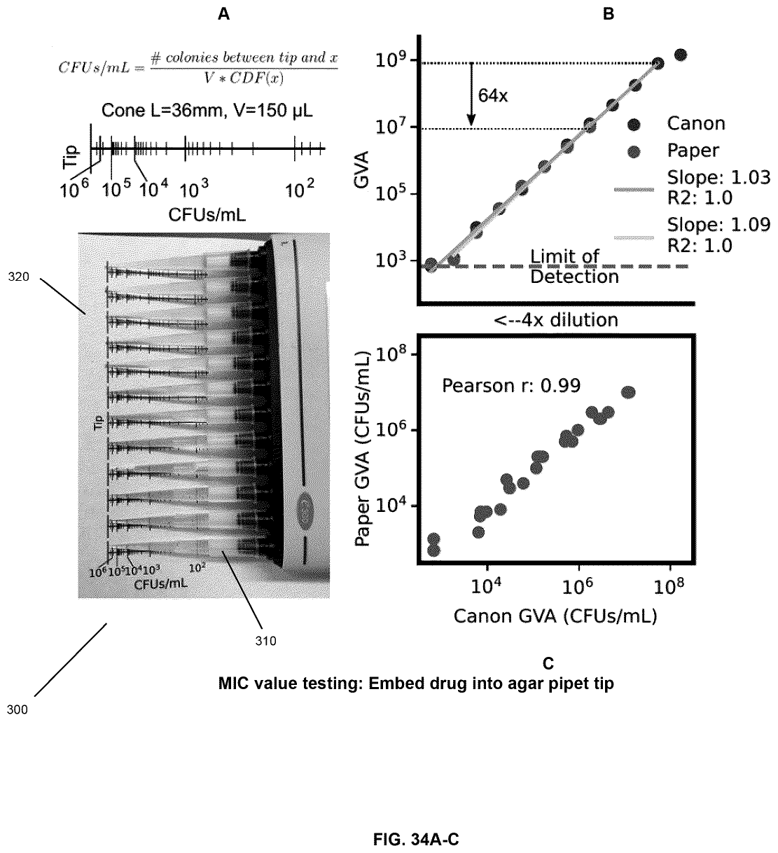

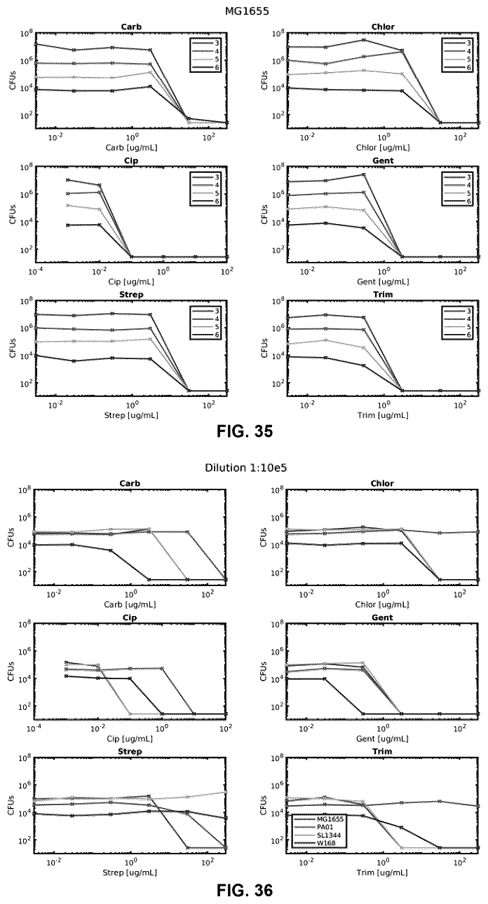

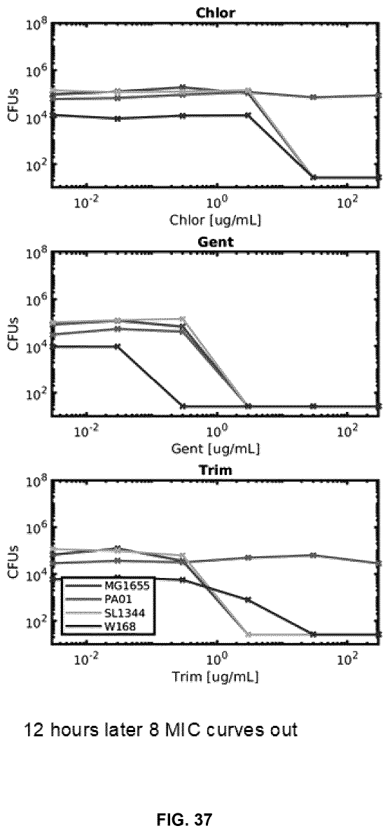

The growth medium containing the diluted sample can be cast into a variable geometry vessel, which as shown in FIG. 24, can include an exemplary axially symmetric three-dimensional pyramid or cone, or within a vessel that forms part of a chip allowing different serial dilutions or culture treatment perturbations. As shown in FIG. 34a, the growth medium containing the diluted sample can be positioned directly within a standard pipette tip that can further be coupled with a standard pipette configured to hold one, or a plurality of adjacent pipette tips. Naturally, such example is exemplary only, as a variety of vessel or pipette holders can be used to secure the vessel and growth medium and allow incubation of the cell sample contained therein. The growth medium suspensions can then be incubated forming embedded colonies within the medium. The time, temperature and other parameter of this incubation step are dependent on the cell to be grown and would be generally understood by those of ordinary skill in the art.

During incubation, viable CFUs, such as microbial CFUs, present in the vessel may form visible embedded colonies that can further be imaged so as to allow their individual identification. In a preferred embodiment, image samples of the three-dimensional volume of the variable geometry vessel may be captured, for example using a light-source and an imager, such as a microscope, digital camera, or cell phone camera that can further digitize and save the captured images for later analysis as further described below. In another example, the captured images of the vessel, can be processed and analyzed to identify a clinically or diagnostically relevant characteristic, such as the presence or absence of a pathogen, change in growth medium, activation of a diagnostic chemical or marker in the growth medium. Naturally, in some instances the identification of one or more of these or characteristics can be visually confirmed by an operator, such as a technical in a laboratory setting or automated detection system as would be understood by those of ordinary skill in the art.

In a preferred embodiment, the captured images of the vessel(s) can be processed and analyzed to identify the number of CFUs along a portion of the length of the variable geometry vessel. In certain embodiments, the number of CFUs can be clinically or diagnostically relevant, while in other embodiments can indicate the effects of a treatment, such as an antibiotic treatment applied to the cells in the sample. In this embodiment, the number of CFUs can be used to determine the MIC of a compound, or the effectiveness of a drug screen that promotes or inhibit growth of the cell as described herein.

In a preferred embodiment, the step of identifying CFUs along a portion of the length of the variable geometry vessel can include the manual or computer-aided identification of individual CFUs from one or more images of portion of said variable geometry vessel, and preferably the terminal portion as shown in FIGS. 1-2, 11, 34. Having identified the number of CFU along a portion of the length of the variable geometry vessel, the number of viable CFUs in the original cultured sample can be calculated and outputted for further analysis and the like. As noted below, due to the novel features of the GVA of the invention, identification of approximately 10-20 colonies' positions, preferably at the tip of the vessel, are required for a reliable estimate using the probability density function as described herein.

As noted above, the GVA of the invention includes embedding a cell capable or reproducing to form an observable colony, such as a bacteria, yeast, fungi or the like, in a vessel having geometrically variable characteristics, namely a 3-D vessel having an axially symmetrical geometry that changes size across all three-dimensions. The higher the concentration of viable cells, the more likely colonies will form in the small volume region at the terminal end, or tip of the variable geometry vessel as compared to the lower cell concentrations. (See e.g., FIGS. 1-2 showing the terminal end of a variable geometry vessel, in this embodiment being a pipette tip) As noted below, the cumulative density function (CDF) distribution of colonies along the vessel can be calculated analytically which can then be used to estimate the initial number of viable cells at the time of embedding using the following function:

$$C D F = \frac{{x}^{3}}{{h}^{3}} and CFU = N ( x ) / CD F ( x ) .$$

Here x is the distance along the ramp from the tip of the vessel, h is the total length of the vessel, sometimes referred to as a “ramp” and N(x) is the number of colonies counted by position x. Thus, by recording all of the colonies and their positions along the vessel, it is possible to estimate the CFUs.

Another aspect of the invention includes an analytical solution that provides a second CFU estimation alternative. Specifically, an operator can: (1) count colonies within any given geometrically variable vessel, and record their positions, (2) use the boundaries of the closest and furthest colonies (limits defined as x1 and x2), and (3) use the probability density function (PDF) to estimate the CFUs using the following function:

$$P D F = \frac{3 * {x}^{2}}{{h}^{3}} and CFU = \frac{\vert x \vert ∈ x 1 \leq x \leq x 2}{{\int }_{x 1}^{x 2} P D F ( x ) d x}$$

Here |x| is the number of colonies found between positions x1 and x2. In practice, the present inventors have demonstrated that recording the x-positions of as few as 10 colonies is sufficient to estimate the CFUs within a sample. It is not necessary to count all the colonies in a dense sample, but only around 10. This insight—counting only colonies within a defined position, referred to sometimes as a specific “wedge”—enables easy measurements of CFUs from the GVA even across the large range of potential values (˜10⁷).

In order to parallelize the GVA of the invention while also establishing an easy protocol, in one embodiment, the present inventors created a pattern of 12 or 48 interleaved, triangular ramps

(FIG. 12). The lower bound on CFU detection is set by the size of the ramps, where larger volumes are able to detect smaller CFU values. The upper bound of CFU is determined by the resolvability of the smallest possible colony within a given wedge. The series of triangular ramps were designed to simplify the mathematics and fabrication. In this embodiment, the triangular ramps have a spacing of 9 mm with a length of 36 mm. The height of the ramp was selected to give a final sample volume of 200 μL, which also sets the lower bound of detection at 5 CFUs/mL. This spacing is convenient to leverage multichannel pipets common in many labs. Current designs hold a 48 well or 12 well chip for 48 or 12 simultaneous experiments, respectively.

In this embodiment, cells are first grown in 96 well plates in the presence of the specific experimental perturbations (e.g. drug treatment) to be measured. After treatment, cells are then mixed in a soft agarose and added to the wells so that the growing colonies are immobilized in a 3D vessel. Tetrazolium chloride (TTC) may be added to the agarose such that metabolizing cells turn red enhancing imaging contrast, though the TTC is unnecessary when measuring yeast colonies given their natural optical contrast. The total time for plating into a 48 well chip (48 experiments) is ˜6 minutes for a single operator, compared to ˜45 minutes in a traditional drop plate assay with the same number of experiments. An example of a potential drug screen is shown in FIG. 12 of 80 compounds, in duplicate, from both stationary and exponentially growing bacteria (384 total tests).

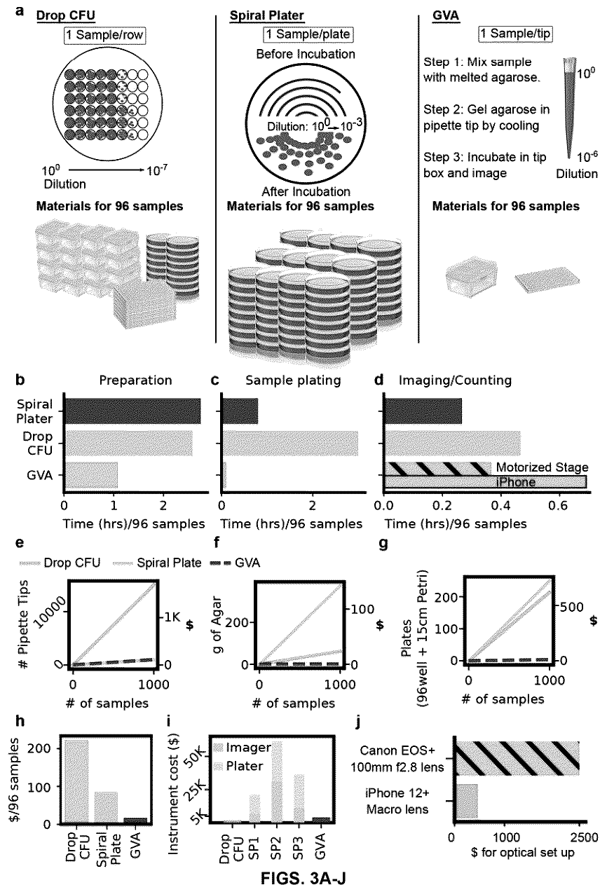

In another preferred embodiment shown in FIG. 3A, the GVA of the invention a sample can be diluted in a growth media and loaded into one or a plurality commercial pipet tips generating a CFU distribution that can mount colonies in such a manner so as to be analytically relevant. In this embodiment, the GVA of the invention can also be used to identify minimum inhibitory concentration (MIC) of a compound in a high-throughput way. Cells can be grown, and treated with specific compound, such as an antibiotic, at a specific concentration that could alter the cell's growth or viability. Individual colonies within the vessel then represent a single cell that was able to grow at the specified concentration, and the MIC can be determined by counting the output colonies. In this embodiment, the operator does is not exposed to potentially dangerous pathogens after they have grown to high density on a plate.

In another embodiment, the GVA of the invention can also be used to identify physical or other modulators of cell growth in a high-throughput way. In this embodiment, cells can be grown, and subjected to a physical treatment, such as UV light or genetic modification, that could alter the cell's growth or viability. Individual colonies within the vessel then represent a single cell that was able to grow after the specified treatment, the overall effects of which can be determined by counting the output colonies.

The imaging of the GVA of the invention could be conducted in a number of different ways, including scanning on a conventional microscope with a low magnification (1×, 2×, 4×) objective or using a consumer-grade camera with a macroscopic scanning stage. The microscope offers the highest possible resolution, and thus resolving the densest samples, whereas the consumer camera offers scanning multiple wells in parallel for the highest experimental throughput.

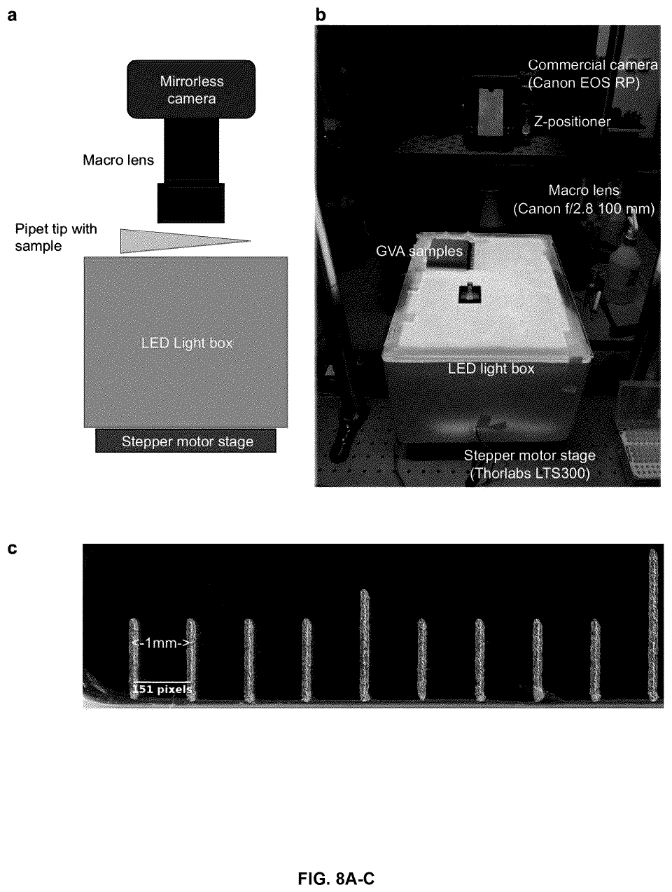

As shown in FIG. 8, initial CFU image data can be captured using an imager, such as a commercially available camera, in this case a DSLR mounted over a custom LED light source. The camera can use a commercial macro lens to achieve the desired spatial resolution. This imaging setup can use lighting from a cyan LED to maximize the contrast of the TTC stain, which thereby enhances colony detection. The light box can move on a computer controlled translation stage (Thorlabs LTS300) which can programmatically move the sample into camera field of view. This imaging embodiment can capture 5 wells per image with an upper resolution of ˜1,000,000 CFUs/mL.

Data acquisition may be controlled by custom scripts in Labview, Matlab, or Python. Outputs from the scripts control the stage, camera, and light source. At each field of view, 2-6 images are taken at slightly varying focal planes (ie. focus stacking). The sample is then moved to a new set of vessels, followed by imaging. This process is repeated through all the vessels, and the software can automatically image up to 4× 48 well chips (192 total conditions) without user input. In a preferred embodiment, the image processing of the GVA of the invention can: (a) create an enhanced image with high signal-to-noise ratio (b) identify individual vessels, and (c) mark the presence and location of individual colonies within a vessel. Using the colony position data, it is possible to calculate the CFUs within a given vessel. All the image processing may be conducted by custom-written scripts in Matlab as described in detail below.

Enhanced focal plane imaging is achieved by combining the 6 acquired images using a focus stacking technique whereby each pixel is selected from the image with the highest contrast, resulting in an extended depth of field. Individual ramps within the vessel are then isolated by using a Hessian transformation followed by a linear convolution to identify the places in the image that mark top, bottom, and sides of each vessel. After separating into individual ramps within the vessel, the colonies are then identified either manually using a mouse click by a user, or automatically using a Hessian transform to identify circular areas. From each colony, x-y coordinates are also calculated and used to estimate the PDF. The GVA of the invention can also be visually processed without the aforementioned image capture and analysis systems. In the embodiment shown in FIG. 34, the GVA of the invention can include a comparative readout system. In this embodiment, a variable geometry vessel, which in this case includes a plurality of pipette tips placed adjacent to one another, can be positioned relative to paper or digital image-based readout CFU indicator (320). As shown in the Figure, the invention can include a GVA assay system (300) including one or more variable geometry vessel (310) containing a incubated cell sample in a growth media and a CFU indicator (320) is precalculated and marked for estimating CFUs. In this embodiment, the CFU indicator (320) of the invention includes a precalculated and marked “ruler” portion calibrated to a 36 mm tip with a agarose volume of 150 μL. Again, as shown in FIG. 34A, a plurality of vessels (310) representing a serial dilution of bacterial samples is overlaid on the paper CFU indicator (320). The CFU estimate is based on the location of the 10^{th} colony counted by the user. FIG. 34b, shows the a comparison between CFU measurements of GCA with a high resolution Canon camera and macro lens (purple) compared to the paper CFU indicator (320) (green) using a basic magnifying glass. As shown, the accuracy is identical with the paper CFU indicator (320), but the maximum number of resolvable colonies is reduced. As further shown in FIG. 34b, a comparison between paper CFU indicator (320) and camera systems (bottom) shows a very high correlation over the dynamic range of the paper based measurement.

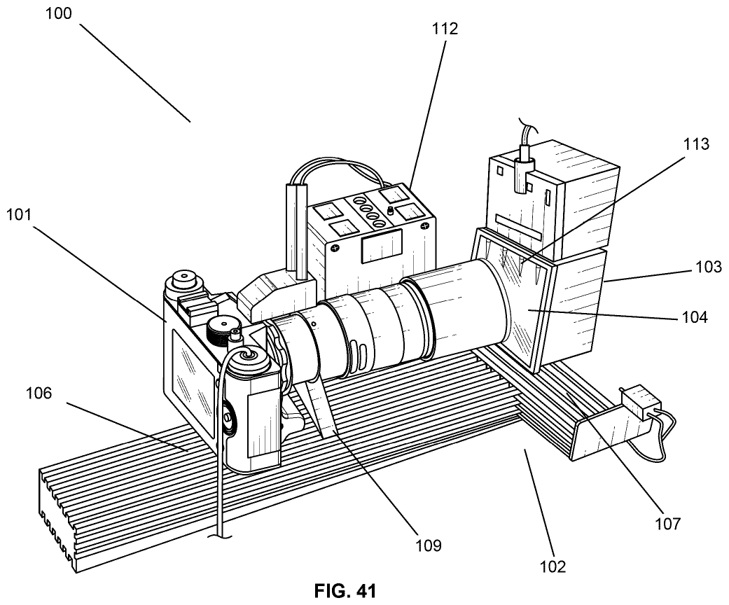

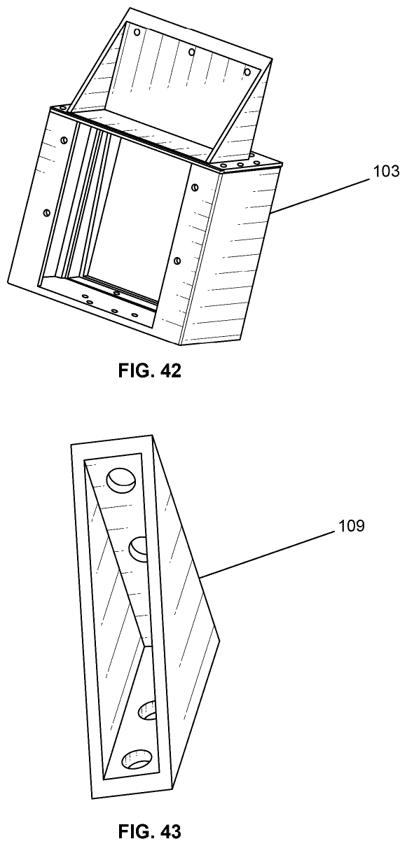

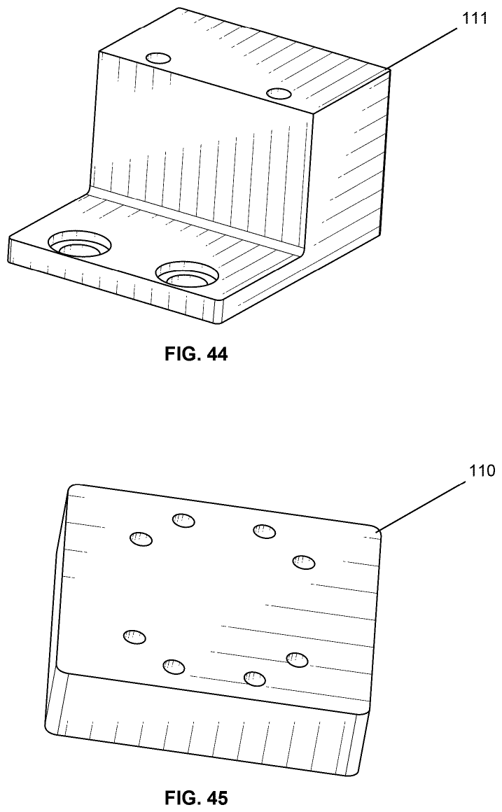

The present invention further includes systems, methods and apparatus for imaging a GVA, preferably using a digital camera. In this embodiment, the GVA imaging system (100) can include an imager (101), which can preferably include a digital camera mounted to a linear stage (102) and further supported by an imager bracket (109). The imager (101) can be positioned adjacent to a frame (103), configured to be coupled to a linear stage (102), and further securing a light source (104). In this configuration, the frame (103) of the invention can secure one or more variable geometry vessels (113) within the imager's (101) field of view. As shown in FIG. 41, a plurality of variable geometry vessels (113) can be coupled with a vessel holder (105) secured to the front of the frame (103) so as to position the vessels (113) between the light source (104) and the imager (101). In this configuration, the imager (101) can capture one or more images of colony-forming units (CFUs) in the vessel, and preferably at the at the terminal portion of the incubated vessel (113) as described herein.

In one embodiment of the invention, the imager (101) can be responsive to a zoom adjustor (107) configured to adjust the position of the imager (101) relative to the frame (103). In a preferred embodiment, the zoom adjustor (107) of the invention can include one or more rails positioned on the linear stage (102) that can allow the imager (101), which is shown here as a camera to be slidably positioned adjacent to the frame (103) and either brough closer, or retracted from the frame (103) adjusting the “zoom” position. of the camera. Such adjustments can be accomplished manually by an operator, or automatically by a controller (112), such as a printed circuit board (PBC) responsive to a processor. In another embodiment, the imager (101) can be coupled with a mounting block (111) that can be responsive to a kill switch to activate, or deactivate, for example the light source (104), and or controller (112).

Again referring to FIG. 41, the frame (103) can be responsive to a pan adjustor (107) configured to allow the horizontal, or “pan” movement of the frame (103) relative to the field of view of the imager (101). In this preferred embodiment, the frame (103) of the invention can be secure to one or more rails on the linear stage (102), preferably through a plate (110). In this configuration, the plate can slide horizontally with respect to the field of view of the imager (101). (Naturally, in certain embodiments the imager (101) of the invention can be responsive to a pan adjustor (107), and the frame (103) of the invention can be responsive to a zoom adjustor (106)). As such, the imager (101) and/or frame (103) of the invention can be independently adjustable so as to position a variable geometry vessel (212) relative to the field of view of the imager (202) allowing it to capture one or more images of colony-forming units (CFUs) embedded in the growth media, preferably at the at the terminal portion of the incubated vessel (212).

The present invention further includes systems, methods and apparatus for imaging a GVA, preferably using a smartphone. In this embodiment, the GVA imaging system (200) of the invention can include an imager (202) positioned adjacent to a light source (not shown). In the preferred embodiment shown in FIGS. 46-47, the imager (202) of the invention can include a smartphone secured to a base (204) such that the smartphone internal camera is positioned approximately vertically and, preferably adjacent to a light source (not shown). An adaptor (206) can be mechanically responsive to the imager (202) of the invention, in this embodiment being a smartphone.

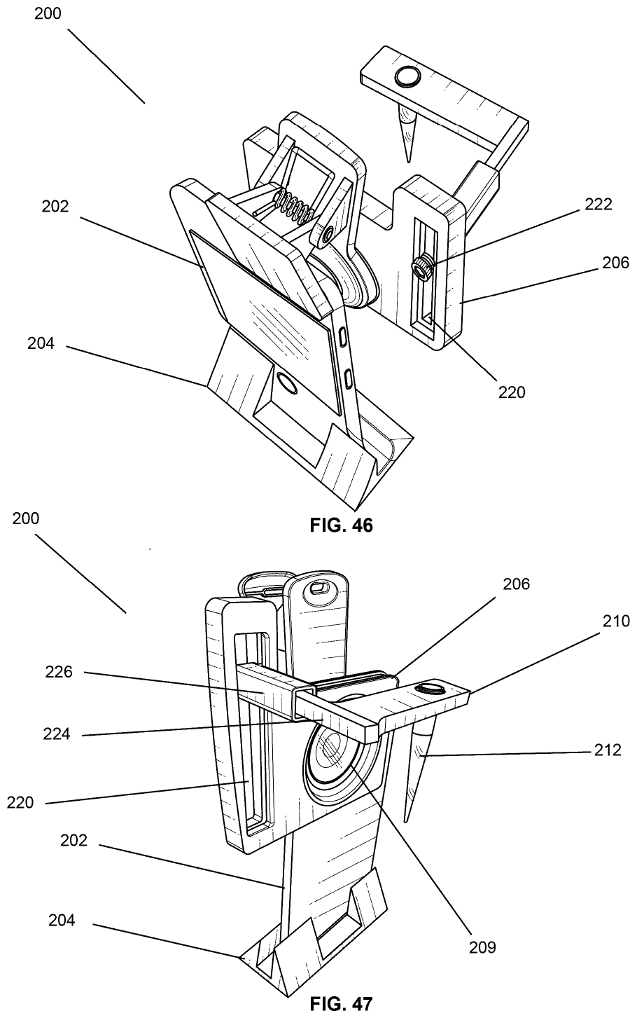

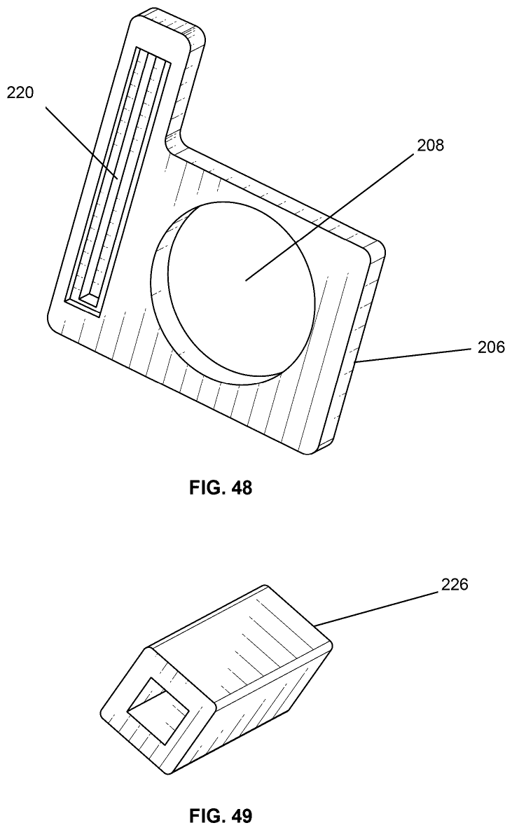

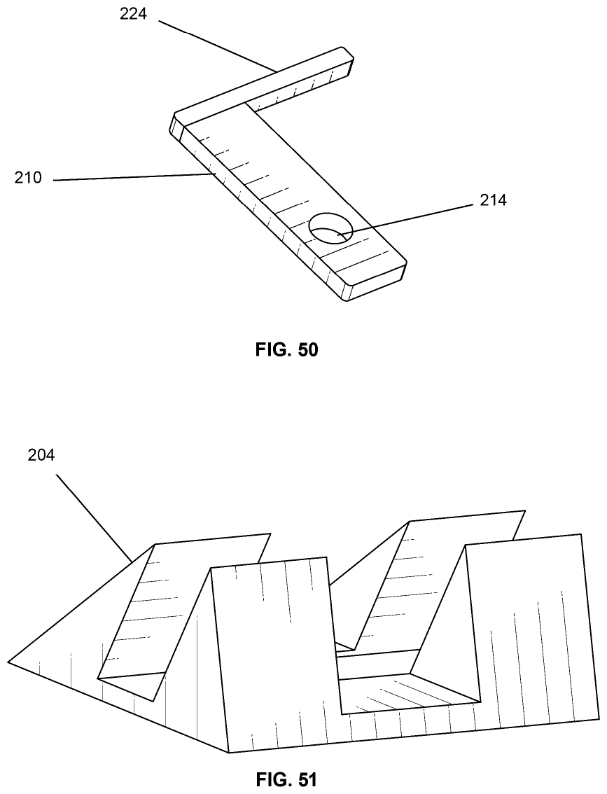

Again shown in FIGS. 46-47, the adaptor (206) of the invention can be coupled with the rear surface imager (202) and positioned over the smartphone's camera which can further be augmented with the placement of a macro lens (209). As shown in FIG. 48, the adaptor (206) can include an aperture lens aperture (208) configured to secure the macro lens (209) of the invention. As shown specifically in FIG. 47, the adaptor (206), having a macro lens (209) secure within the lens aperture (208) can be positioned over the camera of the imager (202), which in this embodiment comprises a smartphone. In this configuration, the smartphone's field of view is augmented by the placement of the macro lens (209) allowing the imager (202) to more accurately capture images of a variable geometry vessel (212) positioned in the camera's field of view.

An axially symmetric variable geometry vessel (212) of the invention can be used to incubate a cell sample (not shown) in a growth media (not shown). Referring again to FIGS. 47-48, in a preferred embodiment, the variable geometry vessel (212) of the invention can be positioned within the field of view of an imager (202), preferably through a mount (210). The mount (210) of the invention can include one, or a plurality of vessel holders (214) configured to secure a variable geometry vessel (212) within the field of view of an imager (202). As shown in the figures, the vessel holder (214) of the invention can include an aperture adapted to fit a variable geometry vessel (212), such as a pipette tip. Naturally, alternative embodiments include additional elements for securing a variable geometry vessel (212), such as a pipette tip in the field of view of an imager (202), such embodiments including couplers, frames, latches and the like. The mount (210) of the invention can be adjustable in a plurality of direction and orientation to allow a user to adjust the position the a variable geometry vessel (212), in relation to the imager (202). In the embodiment shown in FIGS. 47-48, the mount (210) can be mechanically responsive to a depth adjustor (218). In this configuration, the mount (210) may include an arm (224) that can be slidably coupled with a receiver (226) that is further secured to the adaptor (202) of the invention. During operation, the mount (210) can be adjusted so as to alter the depth of the attached vessel (212) with respect to the imager (202).

In the embodiment shown in FIGS. 47-48, the mount (210) can be mechanically responsive to a vertical adjustor (216). In this configuration, the mount (210) can be secured to the adaptor (206) through to a slot (220) positioned approximately adjacent to the field of view of the imager (202). During operation, the mount (210) can be adjusted up or down along a the slot (220) so as to vertically adjust the variable geometry vessel (212) within the imager's (202) field of view. As shown in FIG. 47, the coupler (222) of the invention, shown here in a preferred embodiment as a twist coupler, can be used to secure the vessel (212) in a desired position with the imager's (202) field of view.

In other alternative embodiments, the mount (210) of the invention can be horizontally adjustable, for example through an extended mount surface (210) that can me slidably coupled with the mount (210) and/or arm (224) of the invention. As such, the mount (210) of the invention can be adjustable in multiple orientations so as to position a variable geometry vessel (212) relative to the field of view of the imager (202) allowing it to capture one or more images of colony-forming units (CFUs), preferably at the at the terminal portion of the incubated vessel (212).

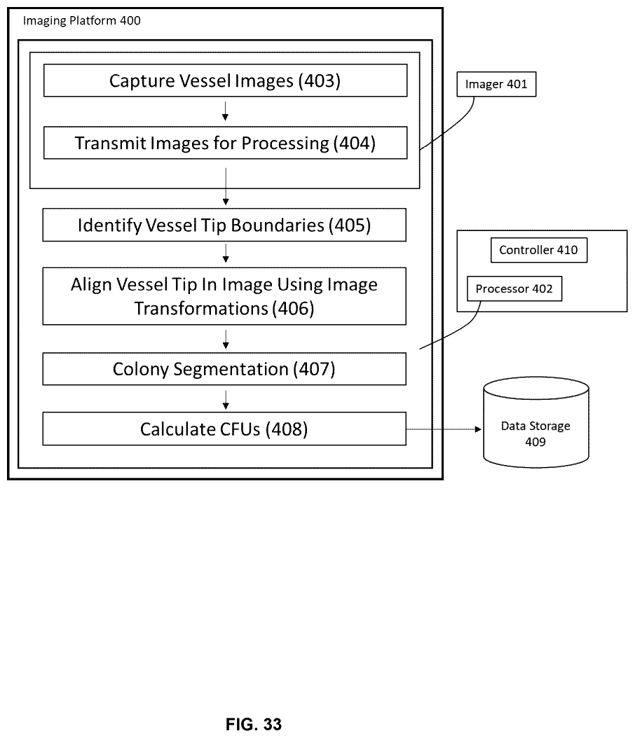

In another embodiment, the invention include novel systems, methods and apparatus to capture and process images of a GVA. In a preferred embodiment shown in FIG. 33, in one embodiment the invention can include imaging platform (400) configured to include a processor 9402) responsive to an imager (401), such as a camera or smartphone. In this embodiment, the imaging platform (400) of the invention can include a computer executable program adapted to capture one or more images of CFUs, preferably at the terminal portion of a variable geometry vessel according to step 403. These images can optionally be transmitted to a separate digital processing device, such as a computer, laptop or tablet having a processor (402) responsive to a controller (410) according to step 404, while in internal embodiments the images capture according to step 403 can be transmitted and process within the imager (401) itself. In this embodiment, the computer executable program can be configured to process the images captured by the imager (401) and: 1) identify the boundaries of the tip of said variable geometry vessel according to step 405; 2) align the tip of said variable geometry vessel using image transformations according to step 406; 3) perform colony segmentation according to step 407; and calculate CFUs according to step 408. All processed images and CFU calculation can be transmitted to a data storage module (409) for further analysis, compilation, or digital storage.

As noted above, methods and systems for identifying and outputting CFU units can be accomplished manually, or through a computer-executable program configured for the same. As a result, the steps of imaging, identifying and calculating an output as herein described may be accomplished in certain embodiments through any appropriate machine and/or device resulting in the transformation of, for example data, data processing, data transformation, external devices, operations, and the like. It should also be noted that in some embodiments, software and/or software solution may be utilized to carry out the objectives of the invention and may be defined as software stored on a magnetic or optical disk or other appropriate physical computer readable media including wireless devices and/or smart phones. In alternative embodiments the software and/or data structures can be associated in combination with a computer or processor that operates on the data structure or utilizes the software. Further embodiments may include transmitting and/or loading and/or updating of the software on a computer perhaps remotely over the internet or through any other appropriate transmission machine or device, or even the executing of the software on a computer resulting in the data and/or other physical transformations as herein described.

Certain embodiments of the inventive technology may utilize a machine and/or device which may include a general purpose computer, a computer that can perform an algorithm, computer readable medium, software, computer readable medium continuing specific programming, a computer network, a server and receiver network, transmission elements, wireless devices and/or smart phones, internet transmission and receiving element; cloud-based storage and transmission systems, software updateable elements; computer routines and/or subroutines, computer readable memory, data storage elements, random access memory elements, and/or computer interface displays that may represent the data in a physically perceivable transformation such as visually displaying said processed data. In addition, as can be naturally appreciated, any of the steps as herein described may be accomplished in certain embodiments through a variety of hardware applications including a keyboard, mouse, computer graphical interface, voice activation or input, server, receiver and any other appropriate hardware device known by those of ordinary skill in the art.

As used herein, a machine learning system or model is a trained computational model that takes a feature of interest, such as the presence of a CFU in a variable geometry vessel and classifies it. Examples of machine learning models include neural networks, including recurrent neural networks and convolutional neural networks; random forests models, including random forests; restricted Boltzmann machines; recurrent tensor networks; and gradient boosted trees. The term “classifier” (or classification model) is sometimes used to describe all forms of classification model including deep learning models (e.g., neural networks having many layers) as well as random forests models.

As used herein, a machine learning system may include a deep learning model that may include a function approximation method aiming to develop custom dictionaries configured to achieve a given task, be it classification or dimension reduction. It may be implemented in various forms such as by a neural network (e.g., a convolutional neural network), etc. In general, though not necessarily, it includes multiple layers. Each such layer includes multiple processing nodes and the layers process in sequence, with nodes of layers closer to the model input layer processing before nodes of layers closer to the model output. In various embodiments, one-layer feeds to the next, etc. The output layer may include nodes that represent various classifications. In certain embodiments, machine learning systems may include artificial neural networks (ANNs) which are a type of computational system that can learn the relationships between an input data set and a target data set. ANN name originates from a desire to develop a simplified mathematical representation of a portion of the human neural system, intended to capture its “learning” and “generalization” abilities. ANNs are a major foundation in the field of artificial intelligence. ANNs are widely applied in research because they can model highly non-linear systems in which the relationship among the variables is unknown or very complex. ANNs are typically trained on empirically observed data sets. The data set may conventionally be divided into a training set, a test set, and a validation set.

Having now described the inventive technology, the same will be illustrated with reference to certain examples, which are included herein for illustration purposes only, and which are not intended to be limiting of the invention.

### EXAMPLES

### Example 1: Rationale and Development of Geometric Viability Assay (GVA)

Standard high-throughput compound screens use growth inhibition, commonly measured using optical absorbance, to quantify a compound's capacity to reduce growth. However, these assays are necessarily blind to phenotypic heterogeneity within a population (e.g., persister cells) as well as the difference between bacteriostatic (i.e., stops growth) and bactericidal (i.e. induces cell death) antibiotics. Therefore, there is an urgent need for assays to identify compounds that are bactericidal, not merely bacteriostatic, in conditions which induce the physiology of persister bacteria (i.e., slow-growing). This need is predicated on a rapid and scalable approach to measuring pathogen viability after drug treatment. The GVA of the invention, therefore, fills a critical gap in current screening technologies for diagnostic and manufacturing applications, as well as biome sampling, and the discovery of novel antibiotic agents against drug-resistant pathogens. The GVA assay can also be used to conduct MIC testing of dangerous pathogens more rapidly and more safely. Additionally, the GVA of the invention addresses a current need to measure the frequency of drug-resistant cells in anti-cancer drug screens with the resolution to identify one in ten million cells that is resistant.

Notably, the analytical framework of the GVA of the invention enabled accurate viability estimates in practice regardless of the optical configuration. In simulations and experiments, errors in the colony count and tip position did not substantially alter CFU estimations when considering the experimental dynamic range. Furthermore, exemplary variable geometry vessels such as pipette tips are not perfect cones; small imperfections in manufacturing were clearly visible at high magnifications. Despite these real world variances-using an imperfect cone, selecting a few colonies, and approximating the tip location-GVA still reproducibly and accurately calculated CFU concentrations across 6 orders of magnitude. This robustness emerges from utilizing the latent information encoded in a colony's position.

Another unexpected feature of the GVA of the invention was the observation of self-limiting colony size depending on the CFU density. As the concentration of colonies increased, the commensurate decrease in colony size preserved colony discreteness even for dense samples. As shown below, colony size, in the strains tested, plateaued after overnight incubation and did not change over several additional days.

The physical restraints of the GVA of the invention results in culturability limitations similar to those found in traditional drop CFU assay [39, 40]. However, Applicant's showed that the GVA of the invention predictably worked for all commonly used laboratory strains tested as well as more complex samples such as biofilms and human-associated biomes samples. Because GVA uses the same growth substrate as traditional 2D culture techniques (e.g. solid media), Applicants, a person of skill in the art would see that the procedures to selectively culture different strains in petri dishes would be transferable to GVA. Finally, the transient thermal shock of the current protocol using agarose did not impact viability of the tested strains, but could be a non-trivial perturbation for certain species. In such alternative embodiments, the use of other hydrogels which crosslink via chemical reaction (e.g. sodium alginate) may be appropriate.

For both the drop CFU and Spiral Plater methods, the incubation time remains a rate limiting step, commonly taking at least overnight for visible colonies to emerge. Similarly, incubation is also a rate limiting step for the GVA of the invention. However, Applicants achieved colony detection across all CFU concentrations within 8 hours for E. coli. This improvement in time to detection is due to the unique optical configuration, the presence of a staining dye, and the 3D geometry which maximizes light scattering. Decreasing time further could be achieved with the use of fluorescent imaging among other alternative embodiments. As such, the GVA of the invention could reduce the time of clinical antibiotic sensitivity profiling among other clinical and/or commercial applications.

In total, Applicants have demonstrated that the GVA of the invention substantially reduced the time and reagents required for measuring cell viability compared to the established drop CFU assay while maintaining the same dynamic range, quantitative nature, and versatility across different species that has made the drop CFU assay the gold standard for viability measurements in microbiology.

### Example 2: GVA Assay Development and Validation

The most time- and resource-intensive step of the classic drop CFU is the dilution series that must be run to count individual colonies across several orders of magnitude. Applicants reasoned the geometry of a cone could create a dilution series in a single step as the cross section at the tip is less than the cross section near the base. Analytically, the probability of a colony forming at any point along the cone's axis proportional to the cross-sectional area at that point (FIG. 1a, cyan circle). This probability is defined as the probability density function (PDF) equal to:

$$PDF ( x ) = \frac{3 * {x}^{2}}{{h}^{3}} \qquad ( 1 )$$

where x is the perpendicular distance from the tip along the x-axis and h is the total length of the cone (FIGS. 1a, 7a-c; see Supplemental Materials for derivation). Equation (1) is applicable for arbitrary cones or pyramids which are axially symmetric (FIG. 7d). The total CFU concentration in the cone can be estimated by:

$$CFUs / mL = \frac{\# Colonies between {x}_{1} and {x}_{2}}{V * {\int }_{x 1}^{x 2} P D F ( x ) d x} \qquad ( 2 )$$

where (x₁,x₂) are the positions of the first and last colony in the counted sub-volume and Vis the volume of the cone. Thus, the highest CFU density resolvable is proportional to the dynamic range of the PDF. In contrast to a variable geometry vessel, such as a cylinder or a wedge, the cone achieves the maximum dynamic range in the PDF by changing shape in all 3 dimensions (FIG. 1b). Importantly, this probability does not depend on the radial (y, z) position of a colony within the cone, only on the perpendicular distance from the tip along cone's axis (x).

Applicants simulated colony distributions in a cone for different CFUs/mL (FIG. 1c,d). As expected, the more CFUs in the cone, the more colonies are found near the tip (FIG. 1d, top panel). The CFUs/mL estimate quickly converges to the correct value (gray dotted line) as more colonies' position are included in Equation (2), regardless of the colony density (FIG. 1d, bottom panel). Remarkably, the CFU estimate is off by less than a factor of 2 from the correct value in 97% of simulations based only on the positions of the first 10 colonies, even if there are over 10,000 colonies in the cone (FIG. 1e, 7f). This rapid convergence to the correct value is the same regardless of the CFU concentration. Therefore, by leveraging the information encoded in the geometry of the cone, it is not necessary to count all the colonies to accurately calculate the colony density. This concept is analogous to a 3D hemocytometer; by counting a subset of colonies within a defined volume, the total concentration can be computed using probabilities.

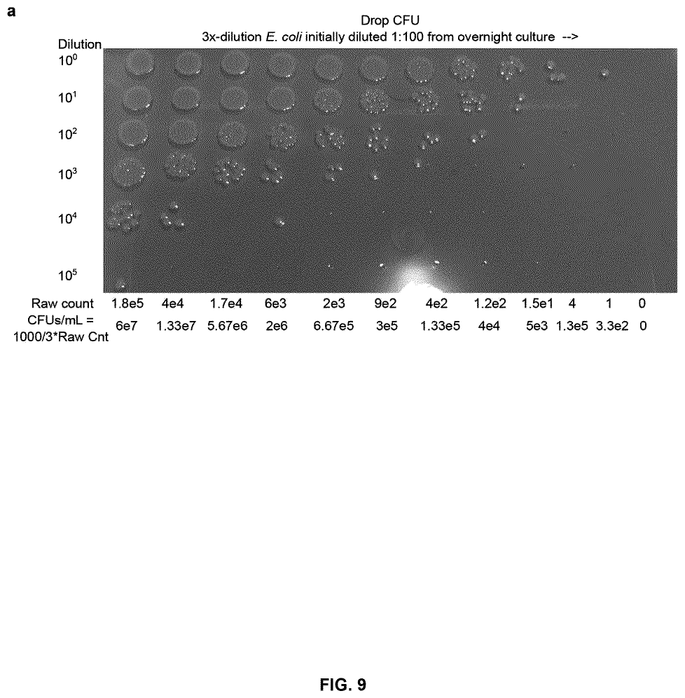

To test the theory, Applicants used a variable geometry vessel in the shape of a cone that is ubiquitous in microbiology-the pipette tip. The first experiment was a dilution series using stationary phase Escherichia coli (BW25113). CFUs/mL of stationary phase E. coli are known to be approximately 10⁹ CFUs/mL after overnight growth. Cells were serially diluted and then each dilution was treated as a sample of unknown concentration of viable cells. Each “sample” was fully mixed with melted LB agarose (cooled to ≤50° C.) to a final agarose concentration of 0.5 %. Triphenyltetrazolium chloride (TTC) was included in the melted agarose to increase the colony contrast. The agarose was allowed to solidify in the tip before the tip was ejected into an empty tip rack (See Methods). The agarose-containing pipette tips were then incubated overnight at 37° C. and imaged the following day using a custom build optical setup with a mirrorless Canon camera (imager) (FIG. 1f, see FIG. 8). In agreement with Applicant's simulations, the distribution of colonies that form in the tip was predictable based on the PDF across >6 orders of magnitude (FIG. 1g, slope ˜1). Remarkably, the final colony size decreased with increasing cell density which prevented colony overlap even at high densities. Comparing the same batch of cells using GVA and the traditional drop CFU assay showed the two approaches are significantly correlated (FIG. 1h, Pearson r=0.98, p-val=4e-16, see FIG. 9 for example drop CFU plate).

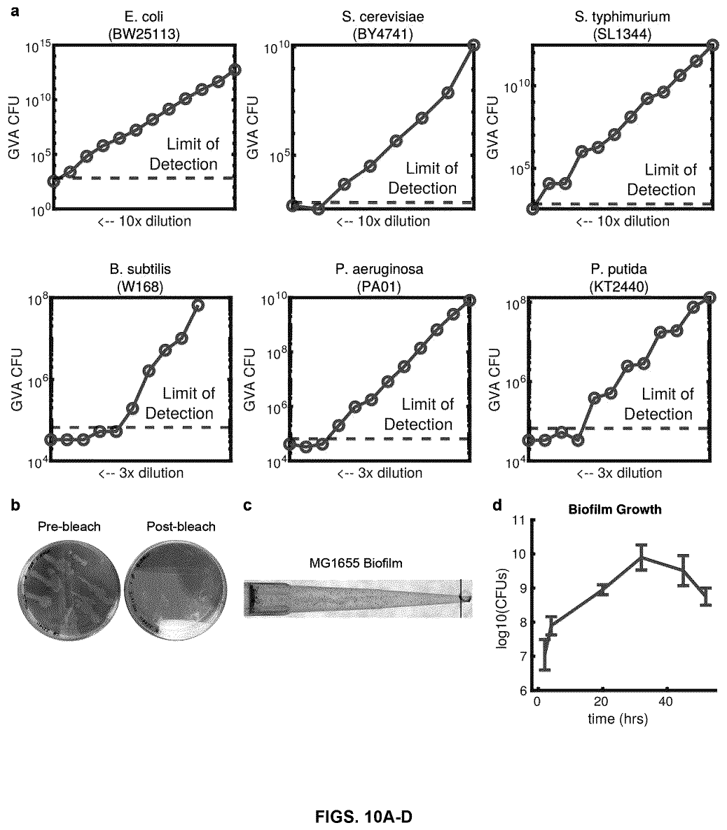

GVA was used to count other gram-negative (Pseudomonas aeruginosa, Salmonella typhimuirium, Pseudomonas putida) and a gram-positive bacterial strain (Bacillus subtilis) as well as cukaryotic yeast cells (Saccharomyces cerevisiae) (FIGS. 1i, 10a). Enclosing the colonies in a pipette tip facilitated handling pathogenic strains because a bleach wash could kill all contaminating cells on the outside of the tip without affecting colony growth inside the tip (FIG. 10b). Viability in E. coli biofilms over time was also tested with GVA (FIG. 10c,d). Finally,

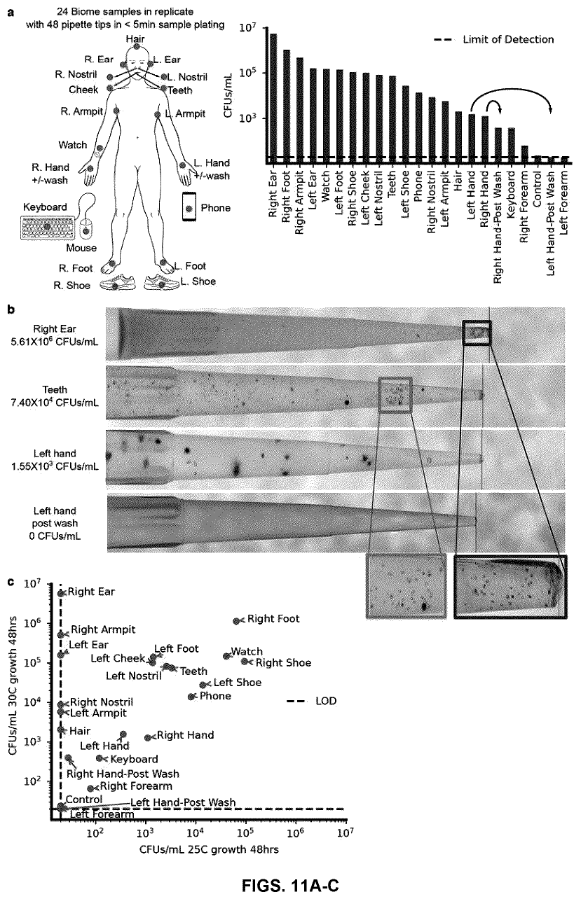

Applicants tested the potential of GVA for rapid quantitation of non-model bacterial species. Human-associated biome viability measurements were conducted using GVA (FIG. 11). Vigorously swabbing 24 locations (FIG. 11a) revealed a large dynamic range of microbial concentrations capable of growth in LB (FIG. 11b). Growing sample replicates at different temperatures revealed temperature-selective growth for different biomes (FIG. 11c). These experiments necessarily underestimate the number of bacteria in these biomes as many human commensals are unculturable. However, because GVA uses solid growth media, the same selective culturing techniques developed over the last 100 years for standard petri dish plating can be leveraged in GVA while also enabling high throughput surveillance of culturable biomes.

Applicants next investigated how the dynamic range and accuracy of GVA depended on the optical configuration using a low cost camera system, also referred to in one embodiment as an imager, which in this case included a smartphone (iPhone®) having a commercial macro lens. Applicants designed a pipette tip holder that positioned a single tip in front of the smartphone (iPhone®) rear camera and a macro lens (FIG. 2a, 13). Calibration revealed the pixel size of the smartphone (iPhone®) was 13.7 microns compared to 6.6 microns for the Canon® EOS® camera with 100 mm f/2.8 macro lens (FIG. 8). Applicants reasoned that the smaller pixel size and lower electron depth in the smartphone (iPhone®) camera would reduce the smallest possible colony detected as compared to the mirrorless camera. As expected, comparing images taken with the camera with the iPhone demonstrated colonies at the highest CFU concentrations were no longer resolvable on the smartphone (iPhone®) (FIG. 2b). Comparing the GVA-calculated CFUs/mL for the same pipette tips of an E. coli dilution series using both the smartphone (iPhone®) and the camera, Applicants measured a reduction in dynamic range of 64× on the iPhone as compared to the Canon camera (FIG. 2c). However, GVA remained highly linear for nearly 5 orders of magnitude (green line, slope=1.04, R²=0.99) with the smartphone (iPhone®) configuration. The correlation between the CFU counts for smartphone (iPhone®) and camera configurations on the same pipette tips was 0.99 (FIG. 2d). Therefore, Applicants found GVA is accurate regardless of the optical configuration, but the dynamic range is set by the maximum camera resolution. The main advantage of GVA is the more than 10× reduction in time, reagent cost, and plastic waste as compared to the drop CFU or Spiral Plater methods (FIG. 3). The Spiral Plater is the most common commercial alternative for the CFU assay utilizing a specialized instrument to dilute the sample along an Archimedes spiral. In order to measure the time savings of GVA, Applicants compared 3 steps of viability assays including the preparation of solid growth media (FIG. 3b), diluting/plating 96 conditions (FIG. 3c), and imaging/counting of the colonies (FIG. 3d). The largest time savings was in the plating step. The drop CFU took 3 hours to manually plate 96 conditions. Current Spiral Plater instruments are reported to take 30 seconds per plate, corresponding to 96 conditions in 48 minutes. GVA took 5 minutes corresponding to a 36× savings in time for plating. GVA was also faster in the time for preparation than both the Spiral Plater and drop CFU approaches. The time for imaging and counting the colonies was the fastest on the Spiral Plater according to the manufacturer-reported time using an automated colony counter. GVA semi-automated colony counting took a similar amount of time to manual colony counting for the drop CFU when including the time for image acquisition, pipette tip segmentation, and user-guided colony detection. In total, using the current instrumentation, a single researcher measured the viability of 1,200 conditions in a day.

Applicants next compared the reagent savings and plastic waste reduction of the three approaches. In the drop CFU assay, since each sample must be diluted and then separately transferred to an agarose pad, 15 pipette tips per sample is standard for our laboratory protocol (FIG. 3e). In GVA, a single pipette tip is used per sample amounting to a 15× savings in pipette tips over the drop CFU (FIG. 3e). In the Spiral Plater assay, a petri dish with solid growth medium is required per condition (FIG. 3g). Compared to the Spiral Plater method, the plastic required is reduced from a petri dish to a pipette tip. Summing the cost of pipette tips, agar, and culture plates at the time of writing, Applicants found the drop CFU was the most expensive in consumables costing an average of $222 per 96 samples compared to the Spiral Plater and GVA which cost $87 and $17, respectively (FIG. 3h). The savings in consumables of the Spiral Plater is offset by the substantial instrument costs (FIG. 3i). Costs were calculated from quotes for 3 Spiral Platers and automated imaging systems solicited from three distributors. The instrument costs for both the GVA and the drop CFU included an electronic, multichannel pipette. Additional instrumentation costs for the GVA depended on the optical configuration (FIG. 3j) which were at least an order of magnitude less than the Spiral Plater systems. In summary, this analysis showed GVA substantially reduced operator time, instrument and reagent costs, and the carbon footprint of viability assays.

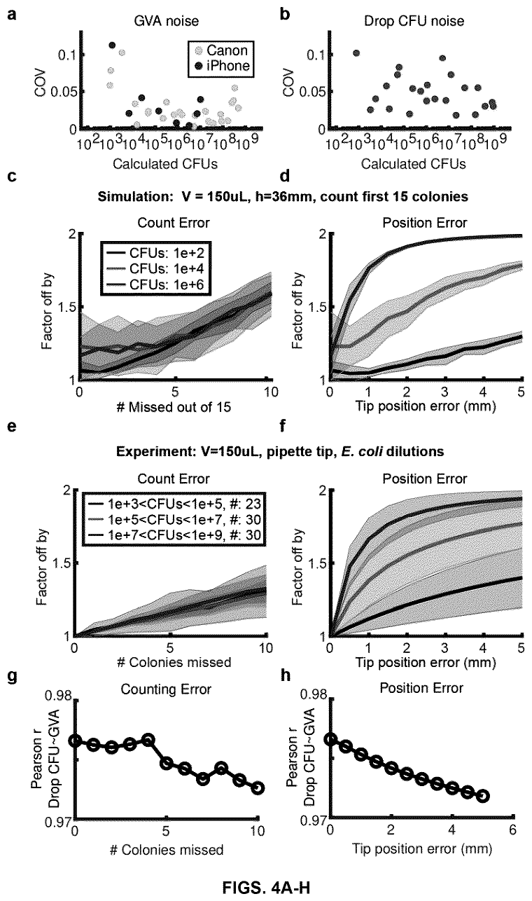

Applicants next investigated the robustness of GVA. Applicants first measured the count noise between 4 technical replicates across CFU concentrations ranging between 10² and 10⁷ CFUs/mL (FIG. 4a,b). Noise was calculated using the coefficient of variation (COV) between replicates. Across all measured CFU concentrations, the GVA noise is less than or equal to the noise of the drop CFU assay for both the camera and smartphone optical configurations. As with the drop CFU assay, the GVA noise is heteroskedastic, increasing as the number of colonies decreases as expected for a Poisson process.

After confirming GVA's low technical noise, Applicants investigated the impacts of two types of real-world errors on GVA calculations: missing colonies and uncertainty in the position of the cone tip. These errors were examined using both simulated and experimental data. Predictably, as the number of missed colonies increases, the error increases (FIG. 4c,e) though the fractional error is the same in all seeding densities. Remarkably, eliminating 10 out of 15 counted colonies in the simulated data resulted in estimates within a factor of 2, regardless of the initial CFU concentration. This robustness was recapitulated in the experimental data and is in agreement with the observation that the position of only 5 colonies is sufficient to calculate the CFUs/mL within a factor of 2 on average (FIG. 1e). For pipette tip position errors, the GVA calculations at high CFU concentrations are more sensitive to misidentification of the tip position than low cell concentrations (FIG. 4d,f blue versus black lines). Nevertheless, missing the tip position by 10% (4 mm for a 36 mm cone) still resulted in an estimate within a factor of 2 from the correct value in both simulations and experiments. Finally, the correlation between the drop CFU and the GVA (FIG. 1h) decreased modestly from 0.98 to 0.97 for combinations of missing up to 10 colonies and missing the tip position by 4 mm (FIGS. 4g,h, 14). These simulated and experimental data highlight the robustness of GVA.

In total, Applicants analyses find GVA is accurate and robust, retaining sensitivity over comparable ranges to the gold standard drop CFU while reducing the cost and time.

### Example 3: High Throughput Viability Screening Against Stationary Phase E. coli.

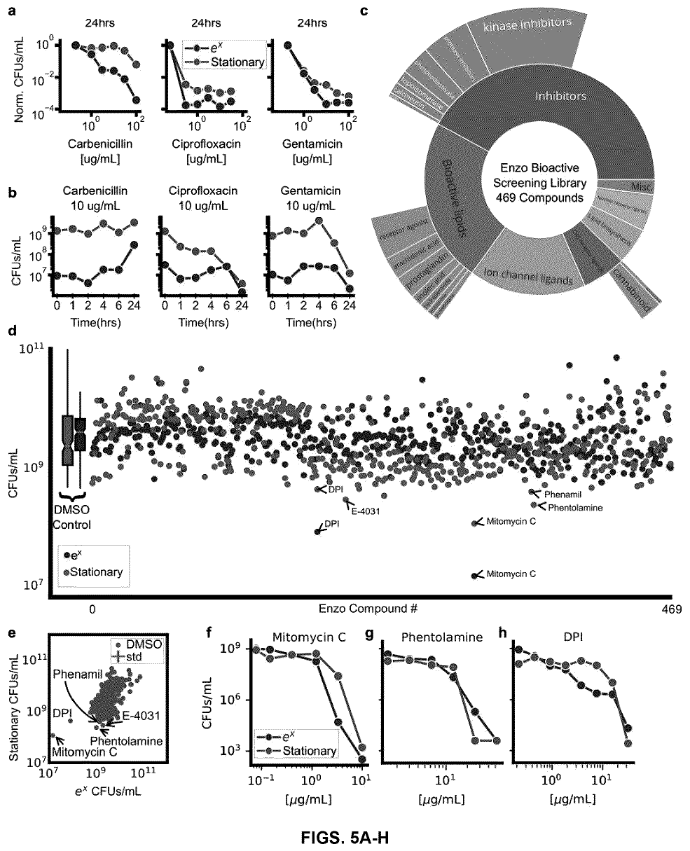

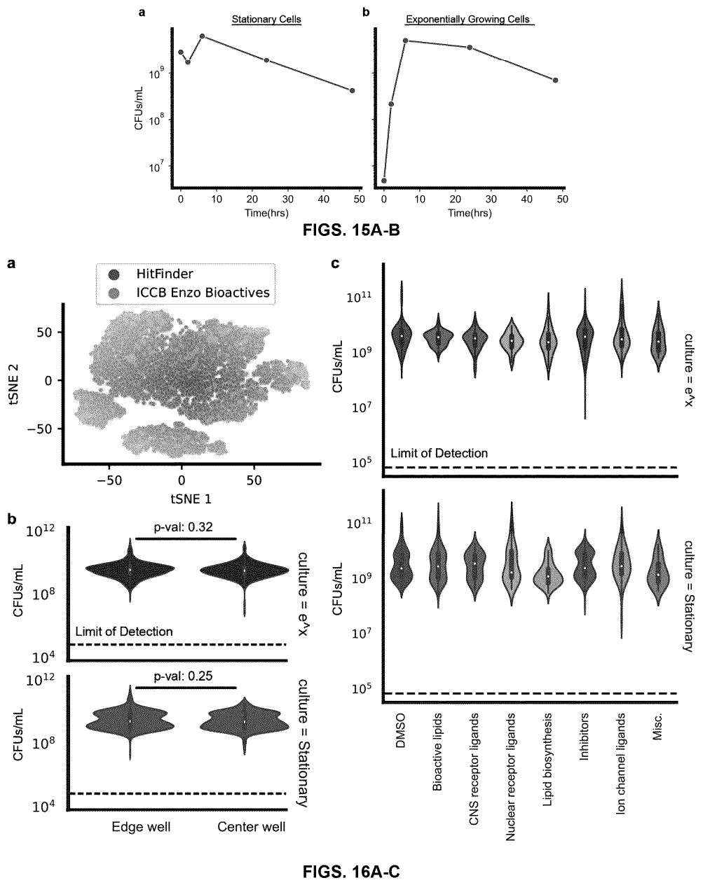

Previous studies have found slow growth is a non-inheritable form of antibiotic tolerance buying time for viable cells to develop genetic resistance. Slow-growing cells commonly have reduced metabolic activity and DNA replication as compared to exponentially growing cells. As a result, slow-growing cells are refractory to antibiotics targeting DNA synthesis (fluoroquinolones), protein translation (aminoglycosides), and cell wall biogenesis (beta-lactams). Growth-dependent tolerance can only be observed by measuring viability but the tedium and cost of the drop CFU assay limits extensive profiling. Using GVA, Applicants directly compared the viability of exponentially growing cells and stationary phase cells to different doses of three antibiotics for varying amounts of time. In total, Applicants tested 3 antibiotics at 6 different concentrations for 5 different durations against stationary and exponential cells, in duplicate, for a total of 360 viability measurements (FIG. 5a,b). This data was acquired by a single researcher in one day using only 4 tip boxes. Stationary phase cells were more resistant to ciprofloxacin, carbenicillin, and gentamicin. Particularly, for carbenicillin, there was less than a 10-fold decline in viability of stationary cells treated with 100 μg/mL carbenicillin for 24 hours, as compared to a 10,000 fold decrease in exponential cells. Treating exponential cells with 10 μg/mL carbenicillin showed no change in the number of colonies during the first 6 hours, followed by an increase in viable cells after 24 hours treatment indicative of a slowly-expanding, drug-tolerant pool (FIG. 5b). Ciprofloxacin at 10 μg/mL had a biphasic pharmacodynamic profile with initial bactericidal activity within an hour resulting in a 10-fold reduction in viability for both stationary and exponentially growing cultures. However, this activity stabilized through 6 hours and a second phase of killing was achieved by 24 hours. Gentamicin at 10 μg/mL required a full 24 hours to achieve more than a 10-fold reduction in stationary phase cell viability. For untreated cultures, Applicants observed the concentration of exponentially growing cells increased till a peak concentration of ˜ 10⁹ CFUs/mL at 6 hours (FIG. 15). Once in the stationary phase, the number of viable cells declined over time as previously reported. These data exemplified the utility of GVA for measuring the efficacy of treatments agnostic to growth rate.

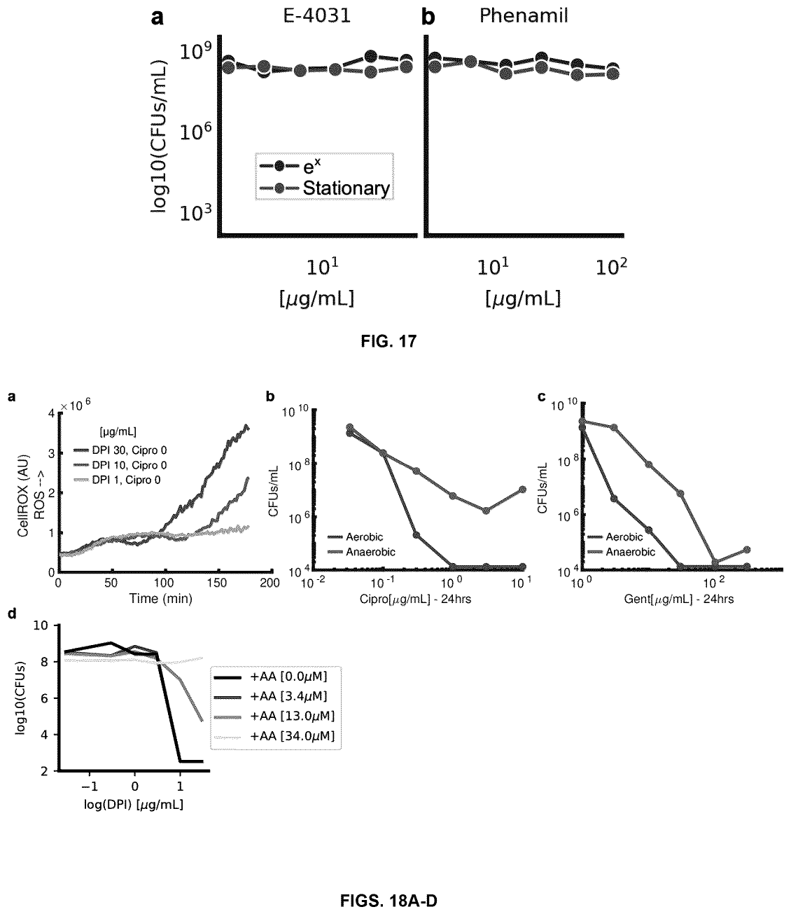

To explore the GVA technique's potential for high throughput viability measurements, Applicants screened the ICCB Enzo Bioactive library (469 compounds) against stationary and exponentially growing cultures (FIG. 5c,d). The Enzo library has a wide breadth of chemical matter including bioactive lipids, small molecule inhibitors, and ion channel ligands (FIG. 5c) and spans the structural diversity of larger libraries like the Maybridge HitFinder library of approximately 14,000 compounds (FIG. 16). The viability of BW25113 E. coli treated with the Enzo library was measured in both exponential and stationary phase. Including controls and removing pipette errors, 2267 conditions were measured. The equivalent screen using the drop CFU or Spiral Plater assays would have required 355 tip boxes or 2267 petri dishes, respectively. GVA required 24 tip boxes. No edge effects were observed for either stationary or exponential plates (Mann-Whitney U test, p-val>0.05, FIG. 16b). Average differences among drug classes were modest (FIG. 16c, p-val>0.001 ANOVA, p-val corrected for multiple hypothesis testing) and none significantly different than the control (p-val 0.01, Pairwise Tukey Test). Five compounds were selected for follow up verification (mitomycin C, phentolamine, E-4031, phenamil, and diphenyliodonium) corresponding to a ˜1% hit rate. Mitomycin C is a known antibiotic acting through DNA cross-linking. As expected, Applicants found it is more active against exponentially growing cells compared to cells in stationary phase (FIG. 5f). Phentolamine is an α-adrenergic receptor antagonist. Phentolamine has previously been shown to block norepinephrine-and epinephrine-induced growth in E. coli putatively by antagonizing α-adrenergic-like receptors. Applicants found at high concentrations (20 μg/mL) stationary cells were more sensitive to the effects of phentolamine than exponentially growing cells (FIG. 5g) corroborating the differential sensitivity observed in the screen. E-4031 and phenamil did not have any dose-dependent effect on viability (FIG. 17). Finally, Applicants found diphenyleneiodonium (DPI), a promiscuous NADPH Oxidase (NOX) inhibitor, to be active against both stationary and growing cultures (FIG. 5h). Previous studies have identified DPI as possessing antimicrobial characteristics; however, the mechanism of DPI bactericidal activity remains unknown. Applicants were intrigued by DPI's bacteriocidal activity as it reduces Reactive Oxygen Species (ROS) in eukaryotes by inhibiting NOXs which is in contrast to the mechanism of many antibiotics which increase ROS pools.

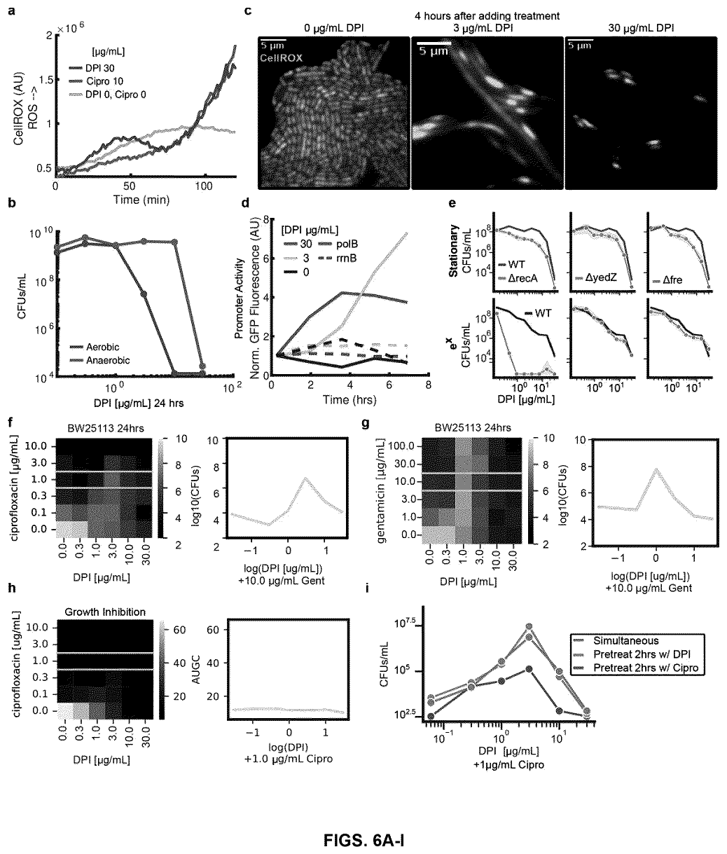

In order to investigate the bactericidal mechanism of DPI, Applicants first examined E. coli ROS levels upon treatment with DPI. ROS levels were determined with the fluorescent CellROX dye which measures cytoplasmic superoxide. Single cell fluorescence was measured over time after treatment with a lethal DPI dose and compared to an untreated control (FIG. 6a). As expected, DPI substantially decreased ROS reaching the nadir around 75 minutes after the drug was added (FIG. 6a compare blue and yellow lines). The depth and duration of the ROS reduction was proportional to the DPI concentration (FIG. 18a). Surprisingly, this decrease was followed by a rapid spike in ROS. In contrast to DPI, ciprofloxacin treatment resulted in monotonically increasing levels of ROS (FIG. 6a, orange line). Increased levels of ROS underlie ciprofloxacin's bactericidal activity; therefore, Applicants next investigated if the ROS spike induced by DPI also underlies its bactericidal activity. Applicants compared DPI sensitivity of stationary phase cells in acrobic versus anaerobic environments. DPI was less active in anaerobic cultures (FIG. 6b), similar to gentamicin or ciprofloxacin (FIG. 18b,c). This data suggested high levels of ROS are part of the bactericidal mechanism of DPI, despite it initially decreasing ROS. In further support of this, adding a ROS scavenger also reduced DPI efficacy (FIG. 18d).

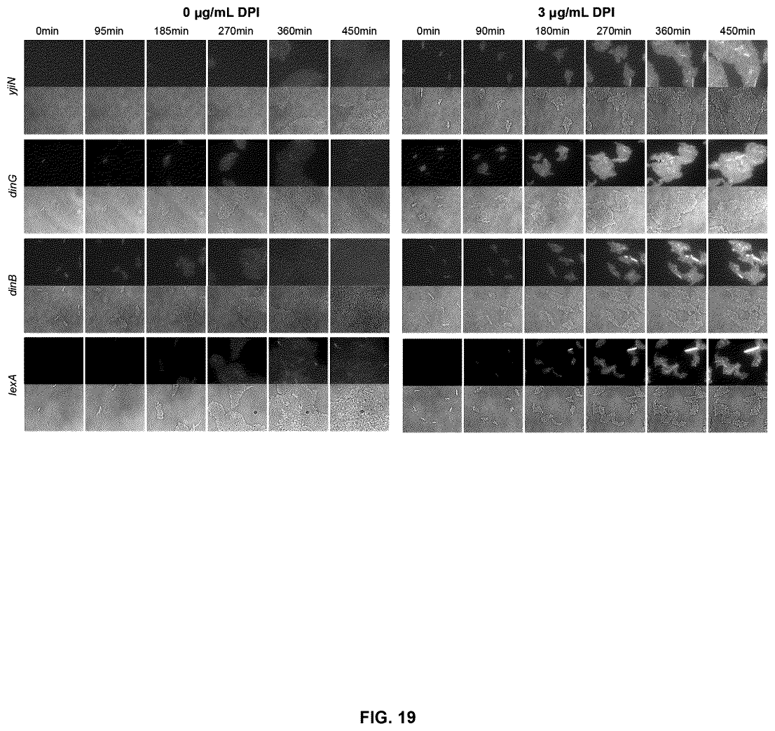

Intermediate DPI concentrations altered ROS levels but maintained viability when measured with GVA. Applicants examined the cell morphology after 4 hours of treatment with less than 10 μg/mL DPI and observed the formation of bacterial filaments (FIG. 6c). Filamentation is a classic hallmark of SOS activation and increases in ROS are an established SOS activator. Therefore, Applicants wondered if DPI was activating SOS. Applicants examined the promoter activity of genes downstream of lexA using the PEC GFP-promoter library. LexA is a master transcriptional repressor of genes in the SOS regulon such as pol, dinB, dinG, and yjiH, and is auto-catalytically degraded by activated recA. Applicants observed persistent, dose-dependent induction of the pol promoter compared to a ribosomal protein control (rrnB) (FIG. 6d, solid versus dashed lines). The highest promoter activity corresponded to an intermediate dose of DPI (3 μg/mL) where filamentation was observed. Applicants also observed a DPI-dependent increase in dinB, dinG, and yjiH promoter activity (FIG. 19). The lexA promoter, which is self-repressed, also increased activity within 90 minutes of DPI addition.

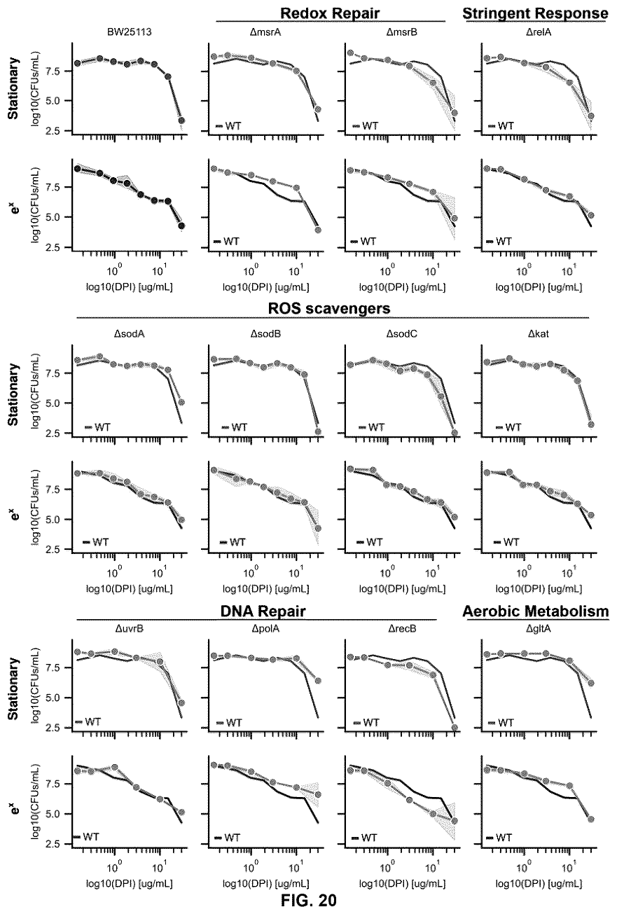

Because SOS activity reduces the efficacy of other bactericidal agents, Applicants predicted that recA-mediated SOS activation was critical for maintaining viability in the presence of DPI. As predicted, recA knockouts were more susceptible to DPI in both stationary and exponential phases of growth (FIG. 6e), though the increased DPI potency was more pronounced in exponentially growing cells. In contrast, knocking out other DNA repair enzymes, redox repair enzymes, or ROS scavengers did not substantially change the potency of DPI in either growth phase (FIG. 20). Knocking out yedZ and fre, genes recently identified as part of a NOX-like system in bacteria, modestly increased the potency of DPI against stationary cells indicating these proteins are unlikely to be the main target of DPI in E. coli (FIG. 6e). Therefore, this data showed DPI activated SOS, and that SOS activation enhanced cell viability.

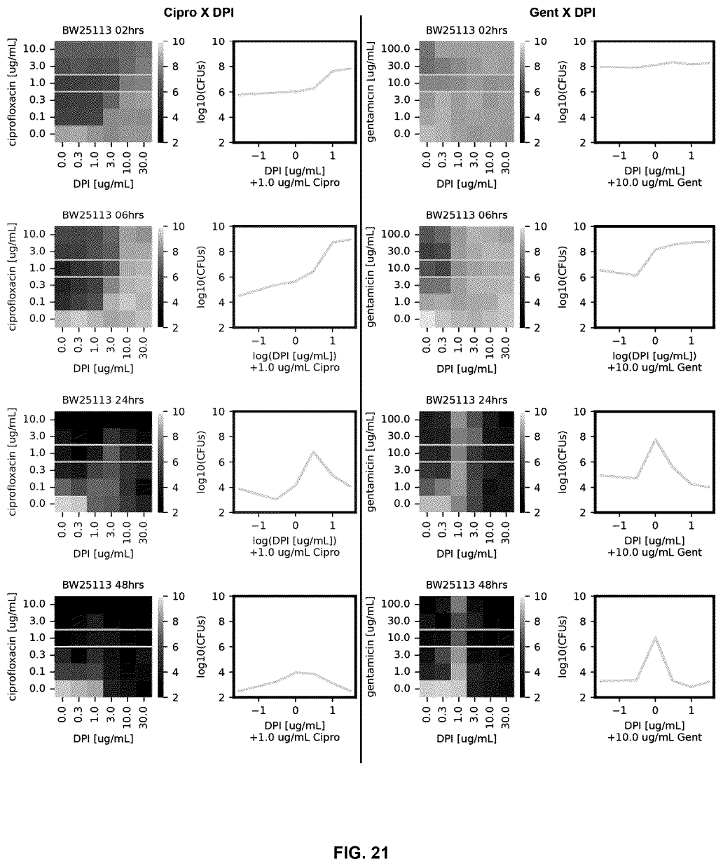

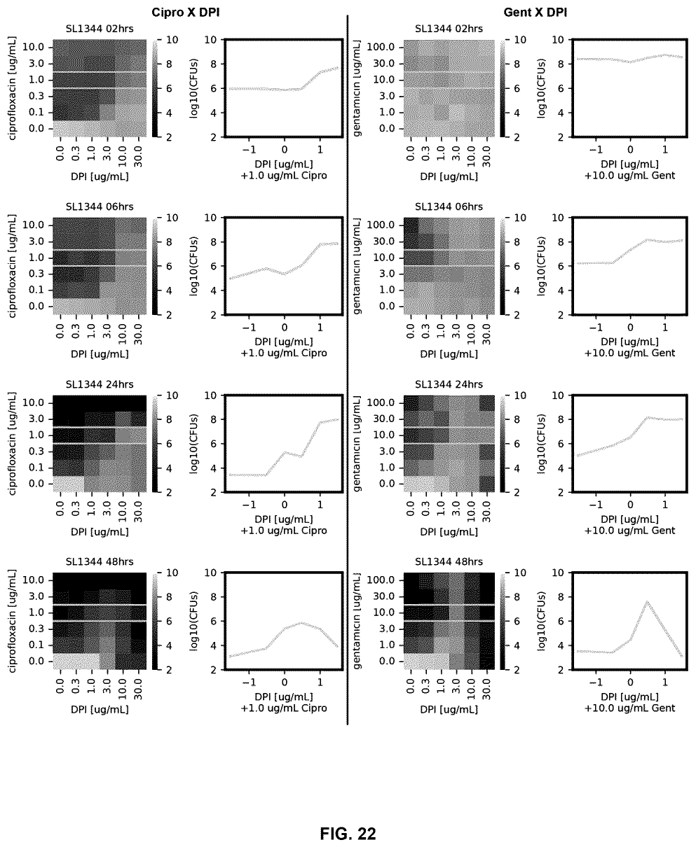

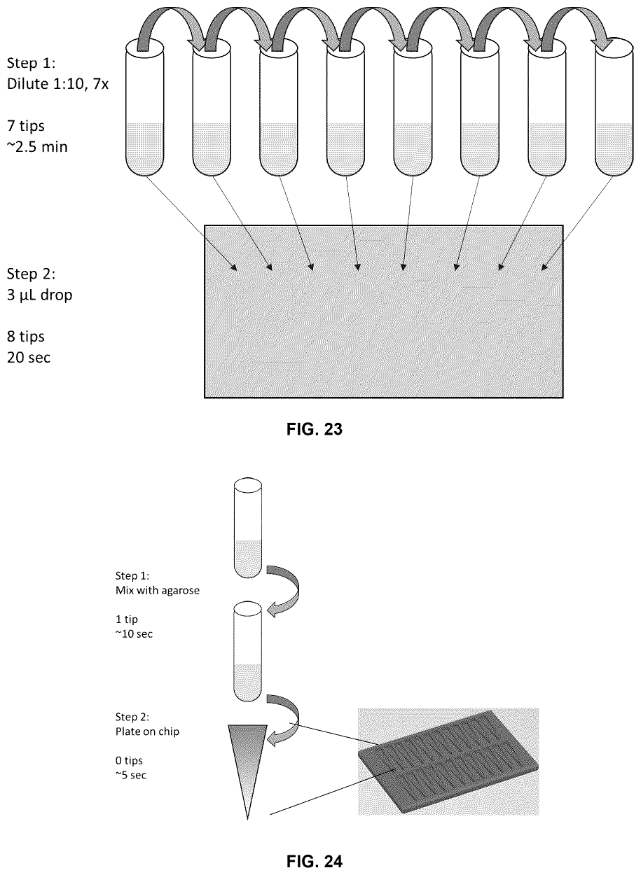

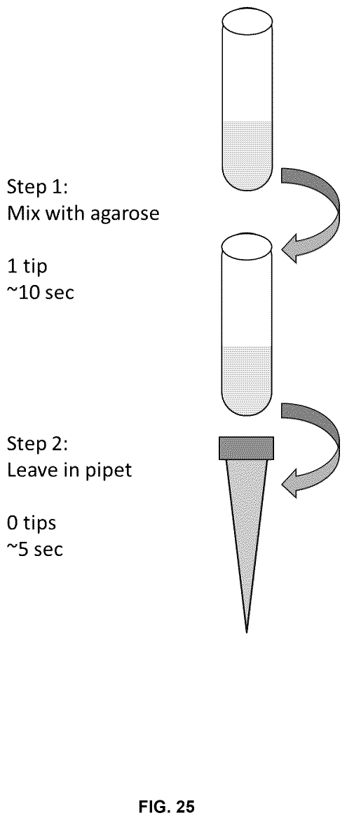

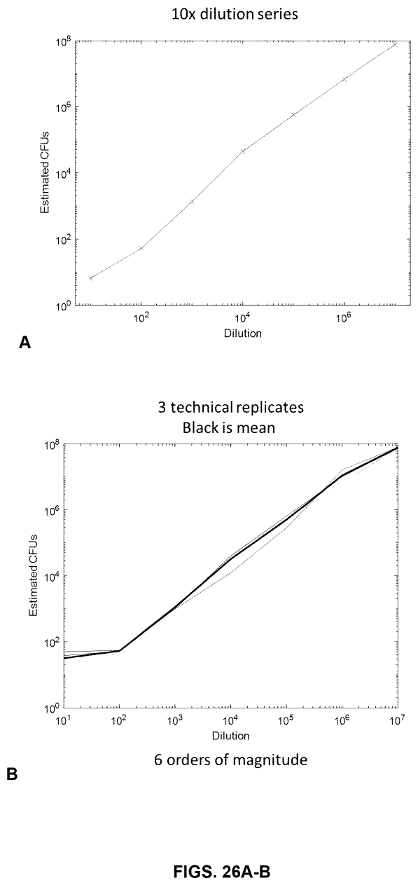

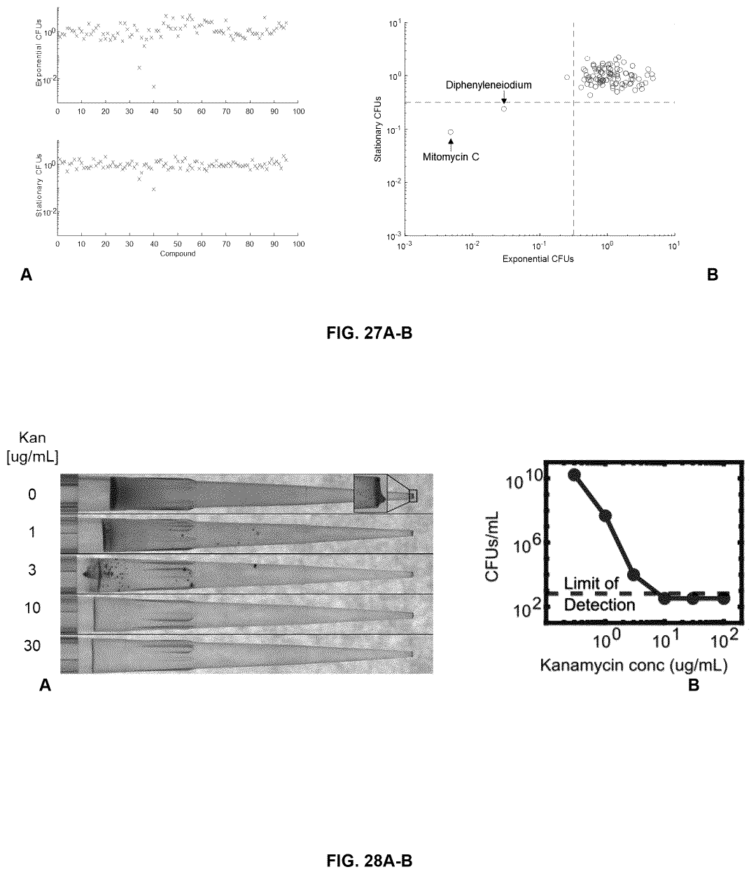

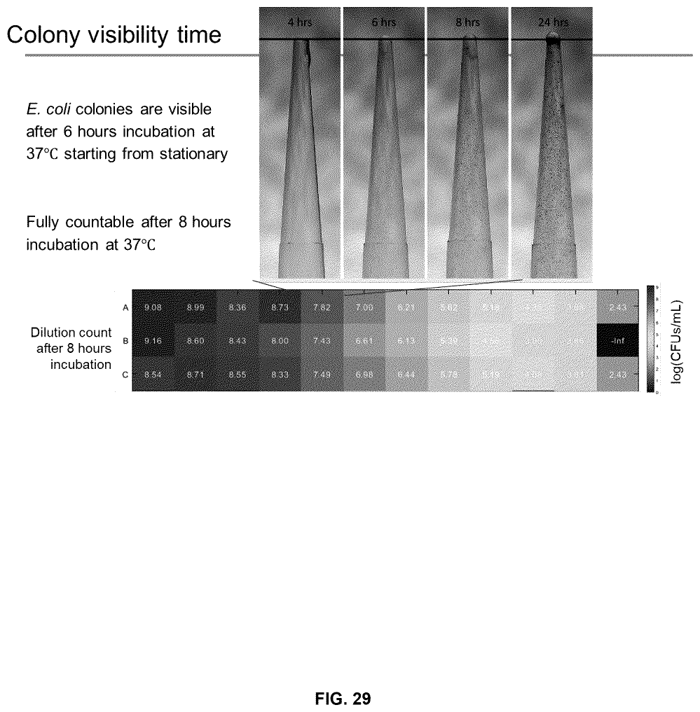

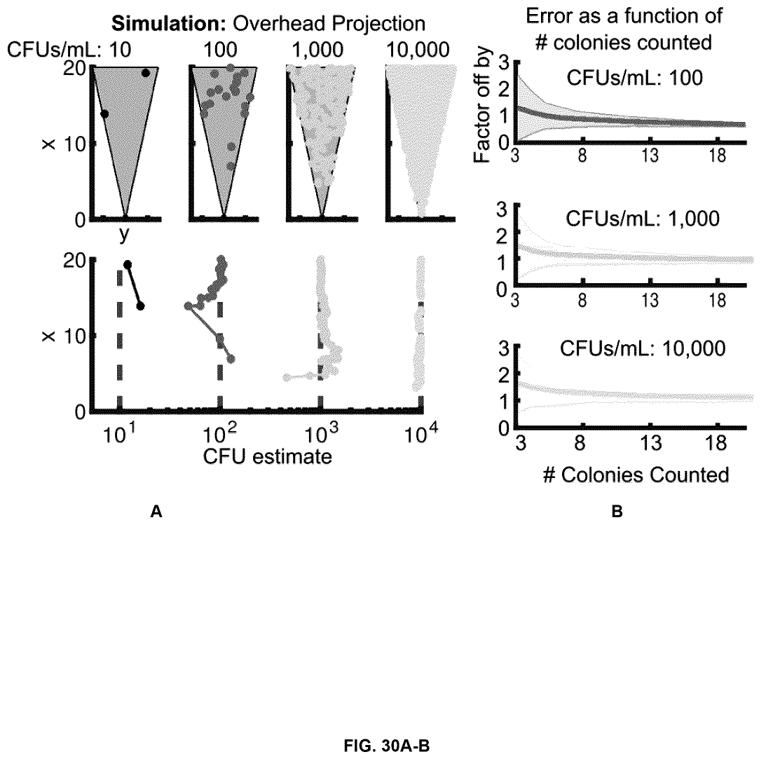

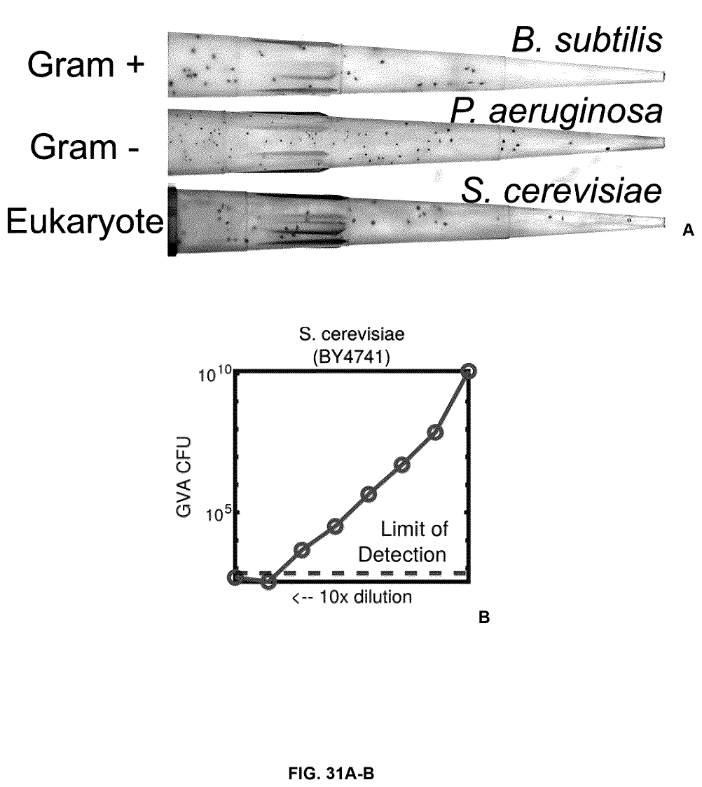

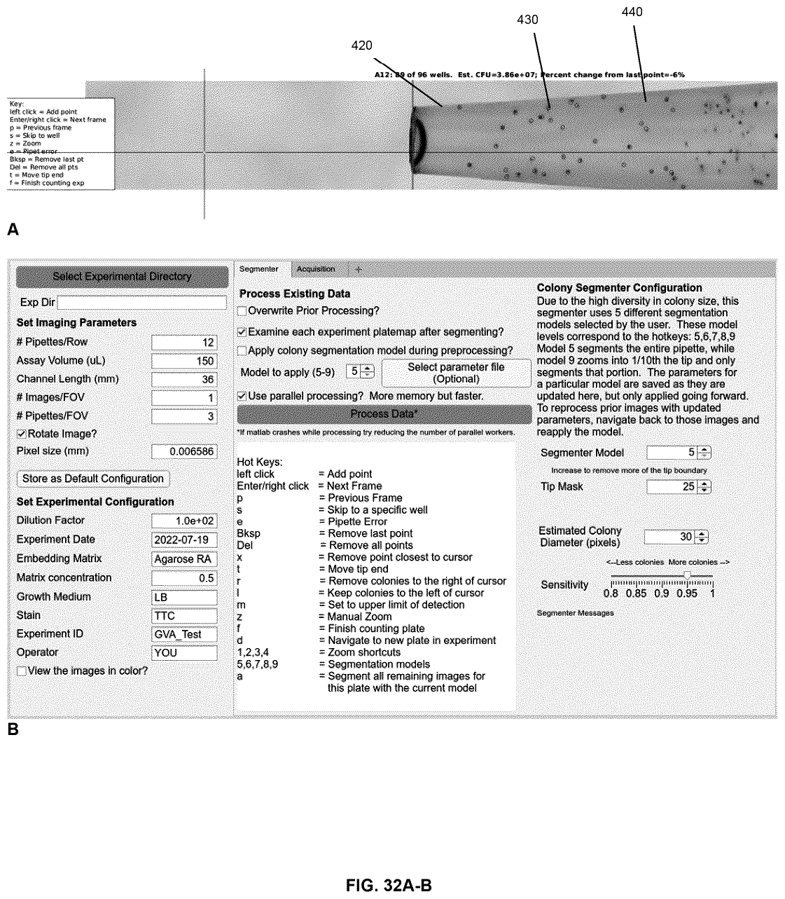

Applicants therefore wondered if DPI would antagonize other antibiotics whose efficacy is reduced by the SOS response. Such antagonism has been observed in combinations of ciprofloxacin with metronidazole, a redox-active prodrug known to activate SOS. To test for antagonism, Applicants measured viability in a time-resolved, checkerboard assay using GVA (FIGS. 6f, 21). In the checkerboard assay, DPI was combined with either ciprofloxacin or gentamicin across a 6×6 dose matrix. The ease of GVA enabled sampling the checkerboard over time, resulting in a complete pharmacokinetic profile of the drug-drug interaction. DPI antagonized both ciprofloxacin and gentamicin against stationary phase E. coli increasing the viability 1,000-fold as compared to either drug alone after 24 hours treatment (FIG. 6f,g). This antagonism was not observed in a growth inhibition assay (FIG. 6h) emphasizing the value of viability data when investigating drug-drug interactions. DPI antagonism of ciprofloxacin and gentamicin was also observed in S. typhimuirium (FIG. 22). Cells pretreated with DPI for 2 hours before adding ciprofloxacin further increased protection, while pretreating with ciprofloxacin for 2 hours reduced DPI's antagonistic effects (FIG. 6i).

In total, Applicants found DPI initially decreased ROS followed by a ROS burst which enhanced its bactericidal effects. As expected with previous studies of ROS lethality, the potency of DPI depended on SOS-activation mediated via recA. By activating SOS, DPI led to an increase in drug tolerance to fluoroquinolones and aminoglycosides as revealed by temporal viability checkerboards.

### Example 4: Materials and Methods

Derivation of the axial probability density function for a cone: Assuming single cells are well mixed before being suspended and cast into a 3D cone, the probability of a colony forming at distance x from the origin is proportional to the percent of the total volume (V) comprised by the infinitesimal volume (dV) at x. dV is defined as:

$$dV = π {{r}^{′} ( x )}^{2} * dx \qquad ( 3 )$$

where r′ (x) is the radius of the circle at x (FIG. 7a, cyan circle). Based on the geometry in FIG. 7a (right panel), Applicants find:

$${r}^{′} ( x ) = \frac{r}{h} * x \qquad ( 4 )$$

where r is the radius of the cone's base and h is the height of the cone.

The probability density function (PDF) for this geometry can be solved for by:

$$C * {\int }_{0}^{h} \frac{π {r}^{2}}{{h}^{2}} {x}^{2} dx = 1 \qquad ( 5 )$$

where C is the normalization constant and is equal to the inverse of the volume V

$$( i . e . C = \frac{3}{π {hr}^{2}} )$$

This leads to the following PDF (FIG. 7b):

$$PDF ( x ) = \frac{3 * {x}^{2}}{{h}^{3}} \qquad ( 6 )$$

The associated Cumulative Distribution Function (CDF) can be found from the integral (FIG. 7c):

$$CDF ( x ) = \frac{{x}^{3}}{{h}^{3}} \qquad ( 7 )$$

Applicants observed here that regardless of the base shape of the cone or pyramid, as long as it is axially symmetric, this PDF holds (FIG. 7d) as a result of the specific geometry of dV canceling out of the PDF due to the normalization constant. Following the same derivation, the PDF of a cylinder is found to be a constant 1/h and the PDF of a 2D wedge is

$$\frac{2 * x}{{h}^{2}}$$

(FIG. 1b). Because of the exponent on x, the PDF of the cone gives the largest dynamic range in the probability (FIG. 1b). The CDF measures the likelihood of having found a colony as function of x if only a single colony is in the cone (FIG. 1b). The PDF is the probability of finding a colony at any point x for only a single colony in the cone. Therefore, there are two equivalent ways of calculating the number of CFUs using either the PDF or the CDF (FIG. 7e).

With the PDF Applicants can estimate the number of CFUs/mL using the equation:

$$CFUs / mL = \frac{\# Colonies between {x}_{1} and {x}_{2}}{V * {\int }_{x 1}^{x 2} P D F ( x ) d x} \qquad ( 8 )$$

where x₁ and x₂ is the position of the first and last colony and V is the cone volume.

With the CDF:

$$CFUs / mL = \frac{\# Colonies between 0 and x}{V * CDF ( x )} \qquad ( 9 )$$

In practice, Applicants find using the PDF estimator to be more convenient because it does not depend on identifying the first colony from the tip, but mathematically these are equivalent.

Strains and growth conditions: E. coli strain BW25113 was used unless otherwise noted in the text. This strain was acquired from the Yale Coli Genetic Stock Center. E. coli was grown in LB (Sigma Aldrich) at 37° C. in a shaking incubator. B. subtilis strain W168 was a kind gift from the Garner lab and was grown in LB at 37° C. in a shaking incubator. P. putida strain KT2440 was a kind gift from Jacob Fenster and was grown in LB at 30° C. in a shaking incubator. S. typhimurium strain SL1344 was a kind gift from the Corrie Detweiler and was grown in LB at 37° C. in a shaking incubator. S. cerevisiae strain BY4741 was a kind gift from Roy Parker and was grown in YEPD at 30° C. in a shaking incubator. P. aeruginosa strain PA01 was a kind gift from the Zemer Gitai and was grown in LB at 37° C. in a shaking incubator. Knockouts were selected from the Keio collection (Dharmacon). The PEC promoter library in E. coli was acquired from Dharmacon (PEC3877).

All bacterial and yeast strains were streaked onto an agar plate with appropriate antibiotic selection if required (kanamycin for Keio and PEC strains). These plates were kept for 1 month in a 4° C. refrigerator. Individual colonies were then selected and grown overnight in 3-5 mL cultures in 12 mL culture tubes with appropriate antibiotic selection if required. Each colony selected was considered a biological replicate. Multiple measurements of the same culture were considered technical replicates.

Antibiotic treatments: Antibiotic treatments were typically performed in 96 well plates with a 12 channel electronic pipette. For stationary phase treatments, bacterial cells were grown overnight (≥16 hours) in a shaking incubator (180 RPM). For P. putida only, cells were grown for 2 days. Upon entering the stationary phase, cells were distributed into a 96 well flat-bottom plates with 100 μL of cells per well. Drug treatments at 1000× were plated into a separate 96 well-round bottom plate. A 100 nL pin transfer was used to dilute the drug plate into the cell plate at a 1:1000 ratio. This plate was then placed into a shaking incubator for the experimental time. To measure antibiotic treatments in the exponential phase, overnight culture was diluted 1:1000 into fresh LB. This culture was then placed into the incubator for 2 hours. After this incubation, the cells were then distributed to the 96 well plate followed by drug treatment.

Drop CFU assay: Drop CFU assays were performed similar to the method described in. Briefly, in a 96 well plate, 90 μL was added to all wells except row A. Into row A, a 100 μL volume of sample solution was added. From row A, 10 μL of cells was taken and added into row B, followed by 3 mixes. This process was repeated from B to C, until the final dilution on row H corresponding to a 1e-7 dilution from the original sample. Pipette tips were changed for each row to reduce sample carry over. From each column of the dilution series, 3 μL drops were transferred onto an LB-agar pad. Once all the liquid was absorbed into the agar (typically 15-30 minutes), the agar plates were inverted and placed into a 37° C. standing incubator overnight. Counting the next morning was performed by hand. The first dilution with individually resolvable colonies was used to count and multiplied by the corresponding dilution factor.

Embedding for GVA: The goal for embedding was to have a uniformly mixed sample in liquid hydrogel that would quickly solidify the 3D mold. Applicants used 0.5% agarose as a convenient hydrogel that would solidify quickly and prevent cell motility once solidified. Pipette tips (200 μL, VWR universal) were most commonly used as a reproducible and cheap 3D geometry scaffold.
1. Preparing the agarose solution. A 0.66% agarose solution was prepared in the cell medium of choice. Applicants found the color of LB and YEPD did not affect the imaging in the pipette tips. Agarose (0.66 g) was added to a 100 mL volume of LB and microwaved until completely dissolved. A careful watch was maintained during the heating to ensure it did not boil over. Upon full dissolution, the liquid was placed in a 50° C. heat bath to maintain in liquid state until ready to use. At this stage, tetrazolium chloride (TTC, 25 μg/ml, final concentration) was added to the LB-agarose from a 1000× stock for all bacteria experiments. Respiring bacteria reduce tetrazolium to water-insoluable formazan which stains the colonies red.
2. Preparing the cells. A fresh 96 round-bottom plate was prepared by adding 50 μL of LB or YEPD to each well. The sample plate with the cells and drugs was removed from the shaking incubator, and a pin transfer tool (2 μL hanging drop, VP409) was used to transfer 2 μL of the treated cells into the 50 μL LB plate. If conducting a time-course experiment, the sample plate was then placed back into the shaking incubator.
3. Embedding. To embed, Applicants found an electronic multichannel pipettor was the most convenient for high numbers of samples. Applicants typically used a 12 channel P200 (Eppendorf explorer, 4861000724). The following items were gathered before pouring the liquid agarose into a reservoir: the 96-well plate with 20 μL samples (from step 2), a box of autoclaved P200 pipette tips, an empty P200 tip box filled with ice water, an empty P200 tip box with 2 mL water in the bottom to hold the embedded cells. At this point, the liquid agarose was poured into a 100 mL reservoir for easy use with the multichannel pipette. Using the pipet and mix function on the pipetor, 150 μL of the LB agarose solution was taken from the reservoir, and mixed twice with 1 row of the sample plate (200 AL final volume, 0.5% final agarose concentration, 1:100 dilution from the sample plate). After mixing, 150 μL was taken into the same pipette tips avoiding bubble formation. These tips were then placed into the ice bath for 6 seconds to ensure the hydrogel was solidified to plug the tip. Then the tips were ejected into the empty pipette tip box. This process was repeated for all 7 additional rows in plate. Using 150 μL and the 1:100 dilution from the original sample gave a lower limit of 667 CFUs/mL.
4. Incubation. Upon completion of the embedding process, the tip box with the LB-agarose-cell suspension is left at room temperature for ˜30 minutes to ensure the agarose is fully solidified. The tips were then moved into a standing incubator overnight for the colonies to grow. Applicants found that the colonies did not change size after overnight incubation so that cells could be imaged up to 4 days post embedding as long as they were maintained in a hydrated environment.

Drug screens: A screen was performed with the ICCB Enzo Bioactive hits library (Enzo, BML-2840-0100). An overnight culture of 60 mL LB was grown to stationary phase with E. coli. The next morning, 60 μL of the overnight culture (stationary phase) was added to a fresh 60 ml, of LB and grown for the 2 hours in the shaking incubator (exponential phase). The cells were then dispensed into 100 μL volumes into 96 well plates.

Biofilm Growth and Treatment: MG1655 E. coli strains were used for biofilms. Overnight cultures were diluted 1:10⁵ in LB. Biofilms were seeded in a U-bottom 96 well plate and grown for 48 hours at 37° C. in a stationary incubator. For temporal experiments, a separate plate was used for each timepoint and biofilms were dispersed at the indicated times. Reported time represent the number of hours after the initial 48 hour incubation. To disperse the biofilms, non-adhered cells were aspirated, wells were washed with PBS, and fresh PBS was added to the wells. The plate was covered with foil plate seals (VWR, 60941-126) and put on a plate shaker at 3000 rpm for 30 minutes. Dispersed cells were diluted 10⁴ and GVA was performed. A crystal violet stain was used to confirm proper dispersal; any replicates that were not fully dispersed were discarded.

Imaging GVA tips: Imaging took place on a custom imaging instrument (FIG. 8) or an iPhone 12 (FIG. 2). For the custom instrument, a mirrorless commercial camera (Canon EOS RP) with a 1:1 macro lens (Canon, f/2.8 100 mm) was used to obtain high quality images that could resolve the smallest colonies. For the iPhone, the parts were designed in FreeCAD and then 3D printed with PLA using a Lulzbot Taz Pro FDM printer. All pieces could fit on the print bed in a single print. Print bed adhesion was increased using a glue stick before printing. The print bed temperature was set to 70° C. for all layers and the nozzle temperature was set to 225° C. Print speed was set to 10 mm/sec for initial layers and then increased to 30 mm/sec for subsequent layers. Post printing, the depth channel (green in FIG. 2a), was tapped with a 8-32 bit. After the holder was assembled on the Xenvo macro lens with the wide field lens removed, the tip was positioned in front of a white backdrop and imaged with ambient illumination using the iphone's autofocus function. 3 images per tip were taken and the tip most in focus was selected before processing using the Matlab app.

The digital camera was mounted above a light box providing even illumination. The light box was then moved by a stepper stage so that 12 tips could be imaged automatically (3 tips per field of view, 4 fields of view). The light box consisted of a Styrofoam box that was covered by a transparent acrylic sheet (McMaster Carr, #8560K257). A white paper was attached to the underside of the acrylic to act as a diffuser. The inside of the Styrofoam box was lined with foil (Reynolds) A high intensity cyan LED (Luxeon Rebel, 3Up) was placed on a heatsink inside the box and was powered with a constant current driver (BuckBlock, 2100 mA). The Styrofoam light box was mounted onto a stepper motor stage (Thorlabs, LTS300). The camera was mounted using the tripod's ¼-20 screw threads onto a z-translator (Thorlabs, MT1) which was affixed to a right angle plate (Thorlabs, AP90). The Z-positioner was used to set a distance such that 3 pipette tips could be imaged in one field of view, and the macro lens was used to bring them into focus. With our camera, this corresponded to a pixel size of 5.8 μm (FIG. 8c). To place the tips onto the light box, a broken 12 channel P200 head was used. This made loading and unloading samples easy using the spring release while also providing a standard orientation for the tips.

Images were collected with a custom Labview script to control the camera and the stepper stage. Labview is called a separate program, digiCamControl (digicamcontrol.com) to access camera functions and acquire images. Typical camera settings used a shutter speed of 1/100s, aperture 6.3, and ISO 100. At each field of view, 5 images were collected followed by a stage movement to the next 3 pipet tips (27 mm). The images were stored directly on the instrument computer as high resolution.jpg files. Using this instrument, a typical experiment of 96 tips could be imaged in ˜7 minutes.

Image processing: The goal of the image processing was to identify and extract individual pipet tips from the collected images and identify individual colonies. These were broken into two steps which were performed sequentially. Matlab (Mathworks, R2021b) was used for all image processing analyses. The developed app can be used without a Matlab license using a compiled version specific to the user's operating system.
1) Pipette tip segmentation: All images from a given field of view were converted to a 16-bit grayscale image. The green channel from the images were summed and that image was used for downstream analyses. The overall orientation of the image was calculated to ensure that each tip was oriented perpendicular to the x-axis. Due to small variations in the tip loading onto the light box, this was necessary to accurately calculate the colony distance from the pipette tip. The Hessian (fibermetric.m) of the image was calculated and convoluted with a horizontal line to locate the angle of the tips. The image was then rotated (imrotate.m) by this angle to orient the pipettes vertically in the image. To identify the x-pixels corresponding to the pipette tip, the Hessian was again calculated from the rotated image. From the middle of the image, a convolution of a single line at different angles was used to calculate the left and right boundaries of the pipette tip. These lines were then extended to the bottom of the pipette tip to locate the left and right boundaries of the tip. Each of the three wells was then saved into a cell array.
2) Semi-automated segmentation: Colonies were segmented using a semi-automated, custom script in Matlab. From the extracted image of the pipette tip, the user selected one of 4 different segmentation routines corresponding to the varying sizes of colonies in the pipette tip. The first routine segmented the entire pipette tip while the last segmentation algorithm zoomed into 1/7th of the full tip and segmented the first 30 colonies. Segmentation was done using Matlab's Image Processing Toolbox. Subsequently, the user could curate the automated segmentation adding missed colonies or removing erroneous colonies.
   The colony count and position of the first and last colonies was used in eqs. (1) and (2) to calculate the GVA estimate of the CFUs/mL. For the error analysis, the factor the GVA estimate differed from the correct value was calculated according to: Factor off by:

$$= \frac{\vert calculated - actual \vert}{actual} + 1 ) .$$

This approach to error calculation takes into account the large dynamic range of possible CFUs/mL.

Microscopy measurements: For all microscopy experiments, cells from overnight cultures were diluted 1:100 in minimal media (PMM) and shaken for 2 hours at 37° C. to ensure cells had exited lag phase. After 2 hours of growth, 2 μL of dilute cell culture was added to the top of a cooled, 200 μL 2% low melt agarose pad with CellROX dye (5 μM). The agarose pad was molded to fit in 96-well square bottom plates (Brooks Automation, MGB096-1-2-LG-L). After 10 minutes of drying, the pad with affixed cells was inverted and pressed into the bottom of an imaging plate. Fields of view (FOV) were selected manually on the microscope. After FOVs were selected and before the imaging started, the drug was added on top as done previously. Applicants have previously found the drug diffuses through the pad on the order of minutes.

Imaging took place using a Nikon Ti2 inverted microscope running the Nikon Elements software package. Fluorescent excitation was achieved with a laser source (488 nm and 561 nm) using a high-angle illumination to minimize the out-of-focus background. All images were acquired with a 40×, NA 0.95 air objective. Images were acquired on an sCMOS camera (Hamamatsu, ORCA-Fusion) camera.

Image processing was done in Matlab (Mathworks, R2020a) and followed the general scheme described in [18]. Briefly, the illumination profile for all images was estimated from the average of 50 images per FOV. Morphological opening and blurring were used to broaden the illumination pattern before correcting the images. After illumination correction, the jitter in the movie was removed by aligning each sequential frame using a fast 2D Fourier transform implemented in Matlab. The background was locally subtracted based on an estimation of the background computed using morphological image opening before segmentation.

Segmenting cells was done using the Hessian-based fibermetric routine implemented in Matlab which is specific for identifying tubular structures. Segmented regions were included only if they met a minimum area and intensity threshold which were manually selected based on the camera and laser settings. To remove rare, segmented debris, the mean Euclidean distance of each cell from all other cells in a multi-dimensional feature space was calculated and objects which were in the 95th percentile or above in average distance were removed. A cell's position in the feature space was defined by its segmented area, perimeter, major/minor axis lengths, and circularity extracted using Matlab's regionprops command.

### REFERENCES

1. Lázár, V., Snitser, O., Barkan, D. & Kishony, R. Antibiotic combinations reduce Staphylococcus aureus clearance. Nature, 1-7 (October 2022).
2. Zheng, E. J., Stokes, J. M. & Collins, J. J. Eradicating Bacterial Persisters with Combinations of Strongly and Weakly Metabolism-Dependent Antibiotics. Cell chemical biology 27, 1544-1552 (December 2020).
3 Hazan, R., Que, Y. A., Maura, D. & Rahme, L. G. A method for high throughput determination of viable bacteria cell counts in 96-well plates. BMC Microbiology 12 (November 2012).
4. Thieme, L. et al. Adaptation of the Start-Growth-Time Method for High-Throughput Biofilm Quantification. Frontiers in Microbiology 12, 2395 (August 2021).
5 Hazan, R., Maura, D., Que, Y. A. & Rahme, L. G. Assessing Pseudomonas aeruginosa Persister/antibiotic tolerant cells. Methods in molecular biology 1149, 699-707 (2014).
6. Otašević, S. et al. Non-culture based assays for the detection of fungal pathogens. Journal de mycologie medicale 28, 236-248 (June 2018).
7 Maturin, L. J. & Peeler, J. T. Bacteriological Analytical Manual (BAM) FDA (2001).
8. Bloom, J. S. et al. Rare variants contribute disproportionately to quantitative trait variation in yeast. eLife 8 (October 2019).
9. Kamrad, S. et al. Pyphe, a python toolbox for assessing microbial growth and cell viability in high-throughput colony screens. eLife 9, 1-24 (June 2020).
10. Kritikos, G. et al. A tool named Iris for versatile high-throughput phenotyping in microorganisms. Nature Microbiology 2017 2:5 2, 1-10 (February 2017).
11. Liu, J., Gefen, O., Ronin, I., Bar-Meir, M. & Balaban, N. Q. Effect of tolerance on the evolution of antibiotic resistance under drug combinations. Science (New York, N.Y.) 367, 200-204 (January 2020).
12. Alves, J. et al. A case report: insights into reducing plastic waste in a microbiology laboratory. Access Microbiology 3, 000173 (October 2020).
13. Choudhry, P. High-Throughput Method for Automated Colony and Cell Counting by Digital Image Analysis Based on Edge Detection. PLOS ONE 11, e0148469 (February 2016).
14. Stokes, J. M. et al. A multiplexable assay for screening antibiotic lethality against drug-tolerant bacteria. Nature Methods 16, 303-306 (March 2019).
15. Scheler, O. et al. Optimized droplet digital CFU assay (ddCFU) provides precise quantification of bacteria over a dynamic range of 6″logs and beyond. Lab on a Chip 17, 1980-1987 (March 2017).
16. Gilchrist, J. E., Campbell, J. E., Donnelly, C. B., Peeler, J. T. & Delaney, J. M. Spiral Plate Method for Bacterial Determination. APPLIED MICROBIOLOGY 25, 244-252 (1973).
17. Finkel, S. E. Long-term survival during stationary phase: evolution and the GASP phenotype. Nature Reviews Micro-biology 4, 113-120 (February 2006).
18. Bruni, G. N. & Kralj, J. M. Membrane voltage dysregulation driven by metabolic dysfunction underlies bactericidal activity of aminoglycosides. eLife 9 (August 2020).
19. Levin-Reisman, I. et al. Antibiotic tolerance facilitates the evolution of resistance. Science 355, 826-830 (February 2017).
20. Leslie, D. J. et al. Nutritional Control of DNA Replication Initiation through the Proteolysis and Regulated Translation of DnaA. PLOS Genetics 11 (July 2015).
21. Zeiler, H. J. & Voigt, W. H. Efficacy of ciprofloxacin in stationary-phase bacteria in vivo. The American Journal of Medicine 82, 87-90 (April 1987).
22. Levin, B. R. & Rozen, D. E. Non-inherited antibiotic resistance. Nature Reviews Microbiology 4, 556-562 (July 2006).
23. Sw, L., Ej, F. & Ja, E. Mode of Action of Penicillin: I. Bacterial Growth and Penicillin Activity-Staphylococcus aureus FDA. Journal of bacteriology 48, 1036 (November 1944).
24. Freestone, P. P., Haigh, R. D. & Lyte, M. Blockade of catecholamine-induced growth by adrenergic and dopaminergic receptor antagonists in Escherichia coli 0157: H7,Salmonella enterica and Yersinia enterocolitica. BMC Microbiology 7, 1-13 (January 2007).
25. Aldieri, E. et al. Classical inhibitors of NOX NAD (P) H oxidases are not specific. Current drug metabolism 9, 686-696 (October 2008).
26. Pandey, M. et al. Diphenyleneiodonium chloride (DPIC) displays broad-spectrum bactericidal activity. Scientific Reports 7, 1-8 (September 2017).
27. Jung, B., Li, T., Ji, S. & Lee, J. Efficacy of Diphenyleneiodonium Chloride (DPIC) Against Diverse Plant Pathogens. Mycobiology 47, 105 (2019).
28. Dwyer, D. J., Kohanski, M. A. & Collins, J. J. Role of Reactive Oxygen Species in Antibiotic Action and Resistance. Current opinion in microbiology 12, 482 (October 2009).
29. Kohanski, M. A., Dwyer, D. J., Hayete, B., Lawrence, C. A. & Collins, J. J. A Common Mechanism of Cellular Death Induced by Bactericidal Antibiotics. Cell 130 (2007).
30. Hong, Y., Zeng, J., Wang, X., Drlica, K. & Zhao, X. Post-stress bacterial cell death mediated by reactive oxygen species. Proceedings of the National Academy of Sciences of the United States of America 116, 10064-10071 (Ma. 2019).
31. Choi, H., Yang, Z. & Weisshaar, J. C. Single-cell, real-time detection of oxidative stress induced in Escherichia coli by the antimicrobial peptide CM15. Proceedings of the National Academy of Sciences of the United States of America 112, E303-E310 (January 2015).
32. Goswami, M., Mangoli, S. H. & Jawali, N. Involvement of Reactive Oxygen Species in the Action of Ciprofloxacin against Escherichia coli. Antimicrobial Agents and Chemotherapy 50, 949 (March 2006).
33. Schoemaker, J. M., Gayda, R. C. & Markovitz, A. Regulation of cell division in Escherichia coli: SOS induction and cellular location of the SulA protein, a key to lon-associated filamentation and death. Journal of Bacteriology 158, 551-561 (1984).
34. Baharoglu, Z. & Mazel, D. SOS, the formidable strategy of bacteria against aggressions. FEMS Microbiology Reviews 38, 1126-1145 (November 2014).
35. Zaslaver, A. et al. A comprehensive library of fluorescent transcriptional reporters for Escherichia coli. Nature Methods 3, 623-628 (August 2006).
36. Podlesek, Z. & Žgur Bertok, D. The DNA Damage Inducible SOS Response Is a Key Player in the Generation of Bacterial Persister Cells and Population Wide Tolerance. Frontiers in Microbiology 11, 1785 (August 2020).
37. Juillan-Binard, C. et al. A Two-component NADPH Oxidase (NOX)-like System in Bacteria Is Involved in the Electron Transfer Chain to the Methionine Sulfoxide Reductase MsrP. Journal of Biological Chemistry 292, 2485-2494 (February 2017).
38. Hocquet, D. & Bertrand, X. Metronidazole increases the emergence of ciprofloxacin-and amikacin-resistant Pseudomonas aeruginosa by inducing the SOS response. Journal of Antimicrobial Chemotherapy 69, 852-854 (March 2014).
39. Cundell, T. The limitations of the colony-forming unit in microbiology. European Pharmaceutical Review 20, 11-13 (2015).
40. Staley, J. T. & Konopka, A. Measurement of in situ Activities of Nonphotosynthetic Microorganisms in Aquatic and Terrestrial Habitats. Annual Reviews in Microbiology 39, 321-346 (November 1985).
41. Bruni, G. N., Weekley, R. A., Dodd, B. J. T. & Kralj, J. M. Voltage-gated calcium flux mediates Escherichia coli mechanosensation. Proceedings of the National Academy of Sciences of the United States of America 114, 9445-9450 (August 2017).
42. Mason, G. & Rojas, E. R. Mechanical compression induces persistent bacterial growth during bacteriophage predation. bioRxiv, 2022.08.12.503793 (August 2022).
43. Meyer, C. T., Jewell, M. P., Miller, E. J. & Kralj, J. M. Machine Learning Establishes Single-Cell Calcium Dynamics as an Early Indicator of Antibiotic Response. Microorganisms 9, 1000 (Ma. 2021).

1. A system for a geometric viability assay (GVA) comprising:
a cell sample introduced to a growth medium;
a variable geometry vessel adapted to incubate said cell sample in said growth medium, wherein the vessel is axially symmetric; and
an imager adapted to capture one or more images of colony-forming units (CFUs) in the incubated variable geometry vessel.

2. (canceled)

3. The system of claim 1, wherein said cell sample comprises a microbial sample selected from: a bacterial sample, a microbiome sample, a yeast sample, and a fungi sample.

4. The system of claim 3, further comprising at least one cell contrasting agent introduced to said growth medium.

5. The system of claim 1, wherein said cell sample comprises a diluted cell sample.

6. The system of claim 1, wherein said growth medium comprises a semi-solid growth medium.

7. The system of claim 1, wherein said variable geometry vessel is selected from, a pipette tip, a translucent variable geometry vessel, a mold having one or more variable geometry vessels, a three-dimensionally-printed chip having a plurality of variable geometry vessels, a wedge-shaped variable geometry vessel, a ramp-shaped variable geometry vessel, or a cone-shaped variable geometry vessel.

8-10. (canceled)

11. The system of claim 1, wherein said growth medium comprises a liquid growth medium for serially diluting said cell sample.

12-14. (canceled)

15. The system of claim 1, wherein said imager is selected from: a microscope, a digital camera, and a smartphone.

16. The system of claim 1, wherein said imager is adapted to identify one or more images of said CFUs at the terminal portion of said variable geometry vessel.

17. The system of claim 1, wherein said imager comprises an imaging platform comprising:
a processor responsive to said imager and a controller;
a computer executable program adapted to identify one or more images of said CFUs at the terminal portion of said variable geometry vessel.

18. The system of 17, wherein said computer executable program is configured to:
identify the boundaries of the tip of said variable geometry vessel;
align the tip of said variable geometry vessel;
perform colony segmentation; and
calculate CFUs.

19. The system of claim 1, further comprising a treatment introduced to said cell sample.

20. The system of claim 19, wherein said treatment comprises one or more compounds that adapted to:
kill the cells in said cell sample;
inhibit the growth of the cells in said cell sample;
alter one or more phenotypic characteristics of the cells in said cell sample; or
alter one or more genotypic characteristics in the cells in said cell sample.

21. The system of claim 20, wherein the treatment compound is selected from: a therapeutic compound, an antibiotic, a bactericidal compound, a bacteriostatic compound, an anti-cancer-compound, an anti-fungal agent, a drug screen compound.

22. The system of claim 17, further comprising an output of the number of viable CFUs in the incubated cell sample.

23. The system of claim 1, further comprising a CFU indicator.

24. A colony-forming unit (CFU) assay device comprising a variable geometry vessel adapted to incubate a cell sample in a growth medium, wherein said variable geometry vessel includes an opening, an intermediate portion being axially symmetrical, and a terminal portion being narrower than said opening.

25. The device of claim 24, wherein said variable geometry vessel is selected from, a pipette tip, a translucent variable geometry vessel, a mold having one or more variable geometry vessels, a three-dimensionally-printed chip having a plurality of variable geometry vessels, a wedge-shaped variable geometry vessel, a ramp-shaped variable geometry vessel, or a cone-shaped variable geometry vessel.

26-30. (canceled)

31. The device of claim 24, wherein said cell sample comprises a microbial sample selected from the group consisting of: a bacterial sample, a microbiome sample, a yeast sample, and a fungi sample.

32. The device of claim 24, further comprising at least one cell contrasting agent introduced to said growth medium.

33-81. (Cancelled)
